(19) United States
(12) Patent Application Publication
(10) Pub. No.: US 2025/0353800 A1
(43) Pub. Date: Nov. 20, 2025

(54) Production of P-Xylene by Liquid-Phase Isomerization and Separation Thereof

(71) Applicant: EXXONMOBIL CHEMICAL PATENTS INC., Baytown, TX (US)

(72) Inventors: Paul Podsiadlo, Spring, TX (US); Robert G. Tinger, Friendswood, TX (US); Xiaobo Zheng, Houston, TX (US)

(21) Appl. No.: 18/872,371

(22) PCT Filed: May 16, 2023
(86) PCT No.: PCT/US2023/022399

§ 371 Date: Dec. 6, 2024

Related U.S. Application Data
(60) Provisional application No. 63/351,898, filed on Jun. 14, 2022.

(51) Int. Cl. C07C 5/27

(52) U.S. Cl. CPC C07C 5/2737; C07C 2529/40; C07C 2529/70

(57) ABSTRACT

A feed mixture comprising one or more xylene isomers may be separated in a p-xylene recovery unit using simulated moving bed, chromatography with toluene as a desorbent to obtain a product stream rich in p-xylene and a raffinate stream lean in p-xylene. Optionally, an intermediate stream may be obtained as well. At least a portion of the raffinate stream or an isomerized raffinate stream may be separated in a distillation column to produce an overhead, stream comprising toluene, which may be fed to the p-xylene recovery unit as at least a portion of the desorbent. If present, the intermediate stream may be isomerized under liquid-phase isomerization conditions and fed to the p-xylene recovery unit. At least a portion of the raffinate stream or one or more lower streams obtained from the distillation column may be isomerized under liquid-phase isomerization conditions and fed to the p-xylene recovery unit.

### CROSS-REFERENCE TO RELATED APPLICATION

This application claims priority to and the benefit of U.S. Provisional Application No. 63/351,898 having a filing date of Jun. 14, 2022, the disclosure of which is incorporated herein by reference in its entirety.

### FIELD

The present disclosure relates to isomerization of C8+ aromatic hydrocarbons and, more particularly, isomerization of C8+ aromatic hydrocarbons under liquid-phase isomerization conditions and separation of p-xylene therefrom.

### BACKGROUND

Worldwide production capacity of p-xylene from various industrial sources is about 40 million tons per year. p-Xylene is a valuable chemical feedstock that may be obtained from C8+ aromatic hydrocarbon mixtures, primarily for conversion into 1,4-benzenedicarboxylic acid (terephthalic acid), which may be used in synthetic textiles, bottles, and plastic materials among other industrial applications. Other xylene isomers experience considerably lower, though significant demand. m-Xylene, for instance, may be utilized as an aviation gas blending component.

C8+ aromatic hydrocarbon mixtures (e.g., o-, m-, and/or p-xylene isomers, as well as ethylbenzene and heavier aromatic hydrocarbons) may be produced through various processes, such as alkylation of lower aromatic hydrocarbons (e.g., benzene and/or toluene), transalkylation, toluene disproportionation, catalytic reforming, isomerization, cracking, and the like. Alkylation of lower aromatic hydrocarbons with methanol and/or dimethyl ether under zeolite catalyst promotion may be a particularly effective and advantageous route for producing p-xylene at relatively high selectivity relative to o- and m-xylene, as described in, for example, U.S. Patent Application Publication 20200308085 and International Patent Application Publication WO/2020/197888, each of which is incorporated herein by reference.

After at least partially separating p-xylene from other C8+ aromatic hydrocarbons, a raffinate stream lean in p-xylene may be obtained. Such raffinate streams may be isomerized to form additional p-xylene and then undergo further separation to isolate the additional p-xylene that has been produced. Conventionally, such isomerization processes have been conducted using vapor-phase isomerization, which is a very energy-intensive process. There has been recent progress in conducting isomerization of xylene isomers by liquid-phase isomerization, which is typically a much less energy-intensive process. Although typically less energy-intensive, the presence of toluene, ethylbenzene, and/or C9+ aromatic hydrocarbons during liquid-phase isomerization may generate unwanted byproducts and result in p-xylene loss or complicated separation thereof.

### SUMMARY

In some aspects, the present disclosure provides processes comprising: (I) providing a feed mixture comprising one or more xylene isomers and optionally ethylbenzene; (II) separating the feed mixture in a p-xylene recovery unit using simulated moving bed chromatography with toluene as a desorbent to obtain a product stream rich in p-xylene, a raffinate stream lean in p-xylene, and optionally, an intermediate stream comprising p-xylene at a concentration higher than in the raffinate stream and lower than in the product stream, the raffinate stream comprising toluene, optionally ethylbenzene, and o-xylene, m-xylene, or any combination thereof; (III) optionally, conducting liquid-phase isomerization under liquid-phase isomerization conditions in the presence of a liquid-phase isomerization catalyst upon at least a portion of the raffinate stream to obtain an isomerized raffinate stream; (IV) separating at least a portion of the raffinate stream and/or, if present, at least a portion of the isomerized raffinate stream in a distillation column to obtain an overhead stream comprising at least a majority of the toluene in the raffinate stream and one or more lower streams each comprising one or more xylene isomers; (V) feeding at least a portion of the overhead stream to the p-xylene recovery unit as at least a portion of the desorbent; (VI) conducting liquid-phase isomerization under liquid-phase isomerization conditions in the presence of a liquid-phase isomerization catalyst and obtaining one or more isomerized recycle streams after conducting the liquid-phase isomerization, the liquid-phase isomerization being conducted upon at least one of: (a) at least a portion of the one or more lower streams; and/or (b) if present, at least a portion of the intermediate stream; and/or (c) the liquid-phase isomerization of at least a portion of the raffinate stream is carried out in (III); and (VII) feeding at least a portion of the one or more isomerized recycle streams to the p-xylene recovery unit.

In some aspects, the present disclosure provides process comprising: (i) providing a feed mixture comprising one or more xylene isomers and optionally ethylbenzene; (ii) separating the feed mixture in a p-xylene recovery unit using simulated moving bed chromatography with toluene as a desorbent to obtain a product stream rich in p-xylene and a raffinate stream lean in p-xylene, the raffinate stream comprising toluene, optionally ethylbenzene, and o-xylene, m-xylene, or any combination thereof; (iii) conducting liquid-phase isomerization under liquid-phase isomerization conditions in the presence of a liquid-phase isomerization catalyst upon at least a portion of the raffinate stream to obtain an isomerized raffinate stream; (iv) feeding the isomerized raffinate stream to a first side of a divided wall distillation column; (v) feeding a portion of the raffinate stream to a second side of the divided wall distillation column; (vi) obtaining a first lower stream rich in p-xylene from the first side of the divided wall distillation column, a second lower stream lean in p-xylene from the second side of the divided wall distillation column, and an overhead stream rich in toluene from the divided wall distillation column; (vii) feeding at least a portion of the overhead stream to the p-xylene recovery unit as at least a portion of the desorbent; and (viii) feeding one or more isomerized recycle streams to the p-xylene recovery unit, the one or more isomerized recycle streams being obtained as at least a portion of the first lower stream.

In still other aspects, the present disclosure provides processes comprising: (A) providing a feed mixture comprising one or more xylene isomers and optionally ethylbenzene; (B) separating the feed mixture in a p-xylene recovery unit using simulated moving bed chromatography with toluene as a desorbent to obtain a product stream rich in p-xylene, a raffinate stream lean in p-xylene, and optionally an intermediate stream comprising p-xylene at a concentration higher than in the raffinate stream and lower than in the product stream, the raffinate stream comprising toluene, optionally ethylbenzene, and o-xylene, m-xylene, or any combination thereof; (C) separating at least a portion of the raffinate stream in a distillation column to obtain an overhead stream comprising at least a majority of the toluene in the raffinate stream and one or more lower streams each comprising one or more xylene isomers; (D) feeding at least a portion of the overhead stream to the p-xylene recovery unit as at least a portion of the desorbent; (E) conducting liquid-phase isomerization under liquid-phase isomerization conditions in the presence of a liquid-phase isomerization catalyst to produce one or more isomerized recycle streams, the liquid-phase isomerization being conducted upon at least one of: (a) at least a portion of the one or more lower streams; and/or (b) if present, at least a portion of the intermediate stream; and/or (c) at least a portion of the raffinate stream; and (F) feeding at least a portion of the one or more isomerized recycle streams to the p-xylene recovery unit.

These and other features and attributes of the disclosed methods and compositions of the present disclosure and their advantageous applications and/or uses will be apparent from the detailed description which follows.

### BRIEF DESCRIPTION OF THE DRAWINGS

The following figures are included to illustrate certain aspects of the present disclosure, and should not be viewed as exclusive embodiments. The subject matter disclosed is capable of considerable modifications, alterations, combinations, and equivalents in form and function, as will occur to one having ordinary skill in the art and having the benefit of this disclosure.

To assist one of ordinary skill in the relevant art in making and using the subject matter hereof, reference is made to the appended drawings, wherein:

FIG. 1 is a block diagram of a system and process for xylene separation and liquid-phase isomerization according to a first embodiment of the present disclosure.

FIG. 2 is a block diagram of a system and process for xylene separation and liquid-phase isomerization according to a second embodiment of the present disclosure.

FIG. 3 is a block diagram of a system and process for xylene separation and liquid-phase isomerization according to a third embodiment of the present disclosure.

FIG. 4 is a block diagram of a system and process for xylene separation and liquid-phase isomerization according to a fourth embodiment of the present disclosure.

FIG. 5 is a block diagram of a system and process for xylene separation and liquid-phase isomerization according to a fifth embodiment of the present disclosure.

FIG. 6 is a block diagram of a system and process for xylene separation and liquid-phase isomerization according to a sixth embodiment of the present disclosure.

### DETAILED DESCRIPTION

The present disclosure relates to isomerization of C8+ aromatic hydrocarbons and, more particularly, isomerization of C8+ aromatic hydrocarbons under liquid-phase isomerization conditions and separation of p-xylene therefrom.

### Definitions

Various specific embodiments, versions and examples of the invention will now be described, including preferred embodiments and definitions that are adopted herein for purposes of understanding the claimed invention. While the following detailed description gives specific preferred embodiments, those skilled in the art will appreciate that these embodiments are exemplary only, and that the invention may be practiced in other ways. For purposes of determining infringement, the scope of the invention will refer to any one or more of the appended claims, including their equivalents, and elements or limitations that are equivalent to those that are recited. Any reference to the “invention” may refer to one or more, but not necessarily all, of the inventions defined by the claims.

In this disclosure, a process may be described as comprising at least one “step.” It should be understood that each step is an action or operation that may be carried out once or multiple times in the process, in a continuous or discontinuous fashion. Unless specified to the contrary or the context clearly indicates otherwise, multiple steps in a process may be conducted sequentially in the order as they are listed, with or without overlapping with one or more other step, or in any other order, as the case may be. In addition, one or more or even all steps may be conducted simultaneously with regard to the same or different batch of material. For example, in a continuous process, while a first step in a process is being conducted with respect to a raw material just fed into the beginning of the process, a second step may be carried out simultaneously with respect to an intermediate material resulting from treating the raw materials fed into the process at an earlier time in the first step. Preferably, the steps are conducted in the order described. However, various steps may occur non-sequentially and/or simultaneously rather than expressly in the order listed.

Unless otherwise indicated, all numbers indicating quantities in this disclosure are to be understood as being modified by the term “about” in all instances. It should also be understood that the precise numerical values used in the specification and claims constitute specific embodiments. Efforts have been made to ensure the accuracy of the data in the examples. However, it should be understood that any measured data inherently contain a certain level of error due to the limitation of the technique and equipment used for making the measurement.

As used herein, the indefinite articles “a” or “an” shall mean “at least one” unless specified to the contrary or the context clearly indicates otherwise. Thus, for example, embodiments using “a fractionation column” include embodiments where one, two or more fractionation columns are used, unless specified to the contrary or the context clearly indicates that only one fractionation column is used.

As used herein, the term “consisting essentially of” means a composition, feed, stream or effluent that includes a given component or group of components at a concentration of at least about 60 wt %, preferably at least about 70 wt %, more preferably at least about 80 wt %, more preferably at least about 90 wt %, or still more preferably at least about 95 wt %, based on the total weight of the composition, feed, stream or effluent.

The following abbreviations may be used herein for the sake of brevity: RT is room temperature (and is 23° C. unless otherwise indicated), kPag is kilopascal gauge, psig is pound-force per square inch gauge, psia is pounds-force per square inch absolute, and WHSV is weight hourly space velocity.

As used herein, “wt %” means percentage by weight, “vol %” means percentage by volume, “mol %” means percentage by mole, “ppm” means parts per million, and “ppm wt” and “wppm” are used interchangeably to mean parts per million on a weight basis. All concentrations herein are expressed on the basis of the total amount of the composition in question. All ranges expressed herein should include both end points as two specific embodiments unless specified or indicated to the contrary.

Nomenclature of elements and groups thereof used herein are pursuant to the Periodic Table used by the International Union of Pure and Applied Chemistry after 1988. An example of the Periodic Table is shown in the inner page of the front cover of Advanced Inorganic Chemistry, 6^{th} Edition, by F. Albert Cotton et al. (John Wiley & Sons, Inc., 1999).

As used herein, the term “hydrocarbon” means (i) any compound consisting of hydrogen and carbon atoms or (ii) any mixture of two or more such compounds in (i). The term “Cn hydrocarbon,” where n is a positive integer, means (i) any hydrocarbon compound comprising carbon atom(s) in its molecule at the total number of n, or (ii) any mixture of two or more such hydrocarbon compounds in (i). Thus, a C2 hydrocarbon can be ethane, ethylene, acetylene, or mixtures of at least two of such at any proportion. A “Cm to Cn hydrocarbon” or “Cm-Cn hydrocarbon,” where m and n are positive integers and m<n, means any of Cm, Cm+1, Cm+2, . . . , Cn−1, Cn hydrocarbons, or any mixtures of two or more thereof. Thus, a “C2 to C3 hydrocarbon” or “C2-C3 hydrocarbon” can be any of ethane, ethylene, acetylene, propane, propene, propyne, propadiene, cyclopropane, and any mixtures of two or more thereof at any proportion between and among the components. A “saturated C2-C3 hydrocarbon” can be ethane, propane, cyclopropane, or any mixture thereof of two or more thereof at any proportion. A “Cn+ hydrocarbon” means (i) any hydrocarbon compound comprising carbon atom(s) in its molecule at the total number of at least n, or (ii) any mixture of two or more such hydrocarbon compounds in (i). A “Cn- hydrocarbon” means (i) any hydrocarbon compound comprising carbon atoms in its molecule at the total number of at most n, or (ii) any mixture of two or more such hydrocarbon compounds in (i). A “Cm hydrocarbon stream” means a hydrocarbon stream consisting essentially of Cm hydrocarbon(s). A “Cm-Cn hydrocarbon stream” means a hydrocarbon stream consisting essentially of Cm-Cn hydrocarbon(s).

As used herein, an “aromatic hydrocarbon” is a hydrocarbon comprising an aromatic ring in the molecular structure thereof. An aromatic compound may have a cyclic cloud of pi electrons meeting the Hückel rule. A “non-aromatic hydrocarbon” means a hydrocarbon other than an aromatic hydrocarbon.

As used herein, the term “lower aromatic hydrocarbons” refers to benzene, toluene, or a mixture of benzene and toluene.

An “effluent” or a “feed” is sometimes also called a “stream” in this disclosure. Where two or more streams are shown to form a joint stream and then supplied into a vessel, it should be interpreted to include alternatives where the streams are supplied separately to the vessel where appropriate. Likewise, where two or more streams are supplied separately to a vessel, it should be interpreted to include alternatives where the streams are combined before entering into the vessel as joint stream(s) where appropriate. Furthermore, a single stream may be split into two or more separate streams and provided to different locations.

As used herein, the term “liquid-phase” means reaction conditions in which aromatic hydrocarbons present in a reactor are substantially in a liquid state. “Substantially in liquid-phase” means ≥about 90 wt %, preferably ≥about 95 wt %, preferably ≥about 99 wt %, and preferably the entirety of the aromatic hydrocarbons, is in a liquid phase.

As used herein, the term “vapor-phase” means reaction conditions in which aromatic hydrocarbons present in a reactor are substantially in a vapor state. “Substantially in vapor-phase” means ≥about 90 wt %, preferably ≥about 95 wt %, preferably ≥about 99 wt %, and preferably the entirety of the aromatic hydrocarbons, is in a vapor-phase.

As used herein, the term “alkylation” means a chemical reaction in which an alkyl group is transferred to an aromatic ring as a substitute group thereon from an alkyl group source compound, such as an alkylating agent. “Methylation” means alkylation in which the transferred alkyl group is a methyl group. Thus, methylation of benzene can produce toluene, xylenes, trimethylbenzenes, and the like; and methylation of toluene can produce xylenes, trimethylbenzenes, and the like.

As used herein, the term “methylated aromatic hydrocarbon” means an aromatic hydrocarbon comprising at least one methyl group and only methyl group(s) attached to the aromatic ring(s) therein. Examples of methylated aromatic hydrocarbons include toluene, xylenes, trimethylbenzenes, tetramethylbenzenes, pentamethylbenzene, hexamethylbenzene, methylnaphthalenes, dimethylnaphthalenes, trimethylnaphthalenes, tetramethylnaphthalenes, and the like.

As used herein, the term “molecular sieve” means a crystalline or semi-crystalline substance, such as a zeolite, with pores of molecular dimensions that permit the passage of molecules below a certain threshold size.

“Crystallite” means a crystalline grain of a material. Crystallites with microscopic or nanoscopic size can be observed using microscopes such as transmission electron microscope (“TEM”), scanning electron microscope (“SEM”), reflection electron microscope (“REM”), scanning transmission electron microscope (“STEM”), and the like. Crystallites may aggregate to form a polycrystalline material. An agglomerate particle comprising multiple crystallites may be present in a material in some cases.

As used herein, the term “rich” or “enriched,” when describing a component in a stream or feed, means that the stream or feed comprises the component at a concentration higher than a source material from which the stream is derived. As used herein, the term “depleted” or“lean,” when describing a component in a stream or feed, means that the stream or feed comprises the component at a concentration lower than a source material from which the stream or feed is derived.

Unless otherwise specified herein, any stream or feed that is “rich” in a particular component may “consist of” or “consist essentially of” that component. A “rich” component of a feed or stream may comprise a majority component of the feed or stream in comparison to other components.

As used herein, the term “overhead stream” refers to a vapor stream that is removed from a top portion of a distillation column.

As used herein, the term “lower stream” refers to a vapor stream or liquid stream that is not an overhead stream and is removed from a location other than a top portion of a distillation column. A “lower stream” may be a side stream or a bottoms stream.

Unless otherwise specified herein, any stream or feed that is “lean” in a particular component may be “free of” or “substantially free of” that component. “Essentially free of” and “substantially free of,” as interchangeably used herein, mean that a composition, feed, stream or effluent comprises a given component at a concentration of at most about 10 wt %, preferably at most about 8 wt %, more preferably at most about 5 wt %, more preferably at most about 3 wt %, and still more preferably at most about 1 wt %, based on the total mass of the composition, feed, stream or effluent in question.

In this disclosure, o-xylene means 1,2-dimethylbenzene, m-xylene means 1,3-dimethylbenzene, and p-xylene means 1,4-dimethylbenzene. Herein, the generic term “xylene(s) or xylene isomer(s),” either in singular or plural form, collectively means one of or any mixture of two through four of p-xylene, m-xylene, and o-xylene at any proportion thereof, and/or ethylbenzene. In the disclosure herein, ethylbenzene is to be considered a xylene isomer. Thus, a mixture of xylene isomers may comprise or consist essentially of one or more of o-xylene, m-xylene, p-xylene, and ethylbenzene. A stream containing xylene isomers may be lean in p-xylene or rich in p-xylene, depending on the location and processing conditions from which the stream is drawn, as explained further herein.

A stream or feed that is lean in one component may be rich in another component. For example, a stream lean in p-xylene may be rich in o-xylene and/or m-xylene.

Liquid-Phase Isomerization Following Separation of p-Xylene from a Feed Mixture

As discussed above, it may be desirable to conduct isomerization following separation of p-xylene from a feed mixture containing C8+ aromatic hydrocarbons. The isomerization may form additional p-xylene from other C8 aromatic hydrocarbons and promote more effective utilization of the feed mixture. Although liquid-phase isomerization may afford benefits over vapor-phase isomerization, such as decreasing energy input requirements, the presence of toluene and/or ethylbenzene during liquid-phase isomerization may generate unwanted byproducts and result in p-xylene loss and/or complicated separation thereof. Description of exemplary liquid-phase isomerization processes, conditions, and catalysts may be found in, for example. U.S. Patent Application Publications 2011/0319688; 201210108867; 2013/0274532; 2014/0023563; and 2015/0051430, the relevant contents of which are incorporated herein by reference. Additional details concerning liquid-phase isomerization processes, conditions, and catalysts are provided herein.

Toluene is often co-present in feed mixtures comprising C8+ aromatic hydrocarbons. Catalysts effective for isomerizing xylene isomers may frequently act upon toluene as well and result in byproduct formation. Advantageously, liquid-phase isomerization catalysts comprising a zeolite having a MEL framework may readily promote isomerization of xylene isomers under liquid-phase isomerization conditions to produce an equilibrium mixture of xylenes from a raffinate stream lean in p-xylene, optionally after further separation thereof. Surprisingly, zeolite catalysts having a MEL framework are substantially inert toward toluene, thereby allowing liquid-phase isomerization to take place even in the presence of high concentrations of toluene and addressing a significant difficulty otherwise associated with liquid-phase isomerization. Although somewhat more active toward converting toluene, zeolite catalysts having a MFI framework may also be used in the advantaged liquid-phase isomerization and further processing operations disclosed herein.

Advantaged processes for separating p-xylene in the disclosure herein may utilize simulated moving bed chromatography, details of which will be familiar to one having ordinary skill in the art. Commercially available simulated moving bed chromatography processes are available from Axens, a French corporation, as ELUXYL® technology, although any other simulated moving bed process may be effectively utilized. By virtue of its lack of reactivity during liquid-phase isomerization, unconverted toluene within a raffinate stream obtained following separation of p-xylene may be isolated and fed to a p-xylene recovery unit, wherein the toluene may advantageously function as a desorbent for simulated moving bed chromatography used therein. Thus, the liquid-phase isomerization and separation processes disclosed herein offer considerable synergy when used for producing p-xylene.

Further advantages of the present disclosure include considerable flexibility in when liquid-phase isomerization is conducted upon the raffinate stream. In some cases, at least a portion of the raffinate stream may be isomerized and recycled to the p-xylene recovery unit without being further separated in a distillation column. Thus, the present disclosure may lower distillation throughput requirements, thereby lowering energy input requirements, and potentially decrease capital equipment costs by facilitating use of smaller distillation columns.

Moreover, the liquid-phase isomerization catalysts and liquid-phase isomerization conditions of the present disclosure also do not lead to significant production of ethylbenzene and other byproducts, which might otherwise complicate further separation operations. To address ethylbenzene or other byproducts that build under the liquid-phase isomerization conditions, the processes disclosed herein may further incorporate vapor-phase isomerization for removing problematic byproducts from a portion of the raffinate stream continually or on an as-needed basis. By coupling vapor-phase isomerization to a liquid-phase isomerization process according to the disclosure herein, the energy input requirements of vapor-phase isomerization may be decreased in comparison to that of processing the entire raffinate stream by vapor-phase isomerization. Additional details and further advantages are discussed in the description that follows.

Before discussing more particular aspects and advantages of the present disclosure in further detail, the processes of the present disclosure will be described with reference to the drawings. In the interest of brevity, common reference characters are used in the drawings to describe elements having similar structure and function in various system and process configurations.

FIG. 1 is a block diagram of a system and process for xylene separation and liquid-phase isomerization according to a first embodiment of the present disclosure. In system and process 100, feed mixture 102, which comprises at least toluene, mixed xylenes and optionally ethylbenzene, is received in p-xylene recovery unit 104. Preferably, the amount of ethylbenzene is below a specified threshold amount and/or feed mixture 102 is processed to remove excess ethylbenzene therefrom, p-Xylene recovery unit 104 utilizes simulated moving bed chromatography with toluene as a desorbent to produce p-xylene product stream 106, which is rich in p-xylene and may consist essentially of p-xylene, and raffinate stream 108, which is lean in p-xylene. Raffinate stream 108 may be rich in at least one of o-xylene and m-xylene and contain toluene and optionally at least some ethylbenzene.

In system and process 100, raffinate stream 108 is provided to distillation column 110 and separated into overhead stream 112 and lower stream 114. A majority of overhead stream 112 comprises toluene, and preferably, overhead stream 112 comprises at least a majority of the toluene within raffinate stream 108 and/or preferably, overhead stream 112 consists essentially of toluene. Overhead stream 112 is fed (recycled) to p-xylene recovery unit 104 as at least a portion of the desorbent used therein. Additional toluene desorbent may be fed to p-xylene recovery unit 104 from an external source (not shown in FIG. 1).

At least a portion of lower stream 114 undergoes liquid-phase isomerization and is fed to p-xylene recovery unit 104 thereafter. As depicted in FIG. 1, lower stream 114 is split into first stream 120 and second stream 122. However, splitting of lower stream 114 into first stream 120 and second stream 122 is optional, depending on factors such as, for example, if byproducts such as ethylbenzene have increased to levels prompting need for removal by vapor-phase isomerization or transalkylation. If produced, second stream 122 may be fed to vapor-phase isomerization unit 130, which conducts vapor-phase isomerization of xylene isomers under vapor-phase isomerization conditions in the presence of a vapor-phase isomerization catalyst, to convert one or more xylene isomers into additional p-xylene. Such vapor-phase isomerization may further convert ethylbenzene into other xylene isomers more effectively than does liquid-phase isomerization, albeit at a higher energy input. Vapor-phase isomerization unit 130 may comprise a portion of a xylenes isomerization loop (not shown), which may further include a distillation column for separating xylene isomers from other aromatic hydrocarbons and a p-xylene recovery unit, which may utilize simulated moving bed chromatography or crystallization recovery technologies.

First stream 120 is fed to liquid-phase isomerization unit 140, which conducts liquid-phase isomerization of xylene isomers under liquid-phase isomerization conditions in the presence of a suitable liquid-phase isomerization catalyst. First stream 120 may be lean in p-xylene but contain other xylene isomers (including ethylbenzene) and residual toluene not separated in overhead stream 112. Under the liquid-phase isomerization conditions in liquid-phase isomerization unit 140, additional p-xylene is produced from the xylene isomers to afford isomerized recycle stream 142 that is rich in p-xylene relative to first stream 120. Isomerized recycle stream 142 is then fed to p-xylene recovery unit 104. As depicted in FIG. 1, isomerized recycle stream 142 is returned to p-xylene recovery unit 104 at a location different from that at which feed mixture 102 is introduced to p-xylene recovery unit 104. It is to be appreciated, however, that at least a portion of isomerized recycle stream 142 may be combined with feed mixture 102 and/or returned to p-xylene recovery unit 104 at the same location where feed mixture 102 is introduced.

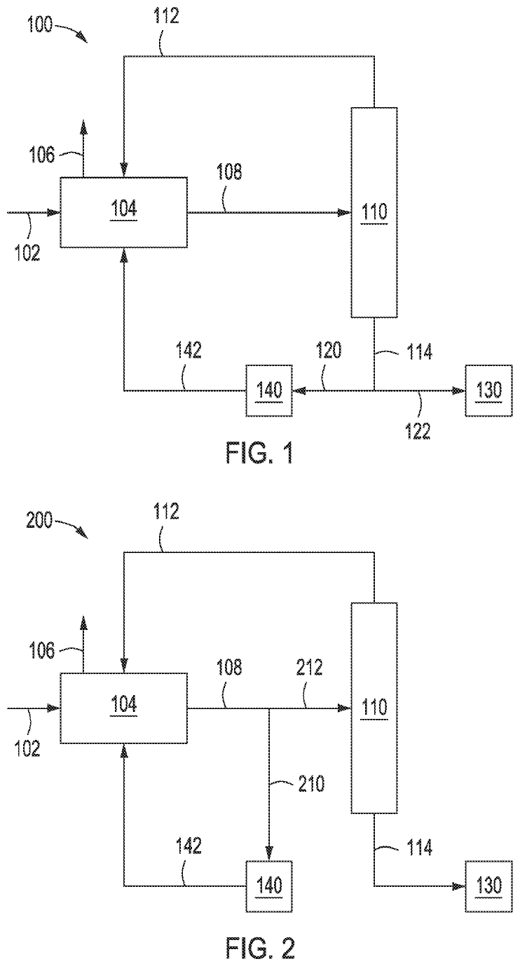

FIG. 2 is a block diagram of a system and process for xylene separation and liquid-phase isomerization according to a second embodiment of the present disclosure. In system and process 200, feed mixture 102 is received in p-xylene recovery unit 104 and is separated into p-xylene product stream 106 and raffinate stream 108 in a similar manner to that described above for system and process 100 in FIG. 1.

In system and process 200, raffinate stream 108 is split into first stream 210 and second stream 212. Second stream 212, containing at least a portion of raffinate stream 108, is provided to distillation column 110 and separated into overhead stream 112 and lower stream 114. A majority of overhead stream 112 comprises toluene, and preferably, overhead stream 112 comprises at least a majority of the toluene present in the portion of raffinate stream 108 within second stream 212, and/or preferably, overhead stream 112 consists essentially of toluene. Overhead stream 112 is fed to p-xylene recovery unit 104 as at least a portion of the desorbent used therein. Lower stream 114 is fed to vapor-phase isomerization unit 130, which conducts vapor-phase isomerization of xylene isomers under vapor-phase isomerization conditions in the presence of a vapor-phase isomerization catalyst, as discussed in more detail above in reference to system and process 100 in FIG. 1.

First stream 210, containing at least a portion of raffinate stream 108, is fed to liquid-phase isomerization unit 140, which conducts liquid-phase isomerization of xylene isomers under liquid-phase isomerization conditions in the presence of a liquid-phase isomerization catalyst. Under the liquid-phase isomerization conditions in liquid-phase isomerization unit 140, additional p-xylene is produced from the xylene isomers to afford isomerized recycle stream 142 that is rich in p-xylene relative to first stream 210. Isomerized recycle stream 142 is then fed to p-xylene recovery unit 104. As depicted in FIG. 2, isomerized recycle stream 142 is returned to p-xylene recovery unit 104 at a location different from that at which feed mixture 102 is provided to p-xylene recovery unit 104. It is to be appreciated, however, that at least a portion of isomerized recycle stream 142 may be combined with feed mixture 102 and/or returned to p-xylene recovery unit 104 at the same location where feed mixture 102 is introduced.

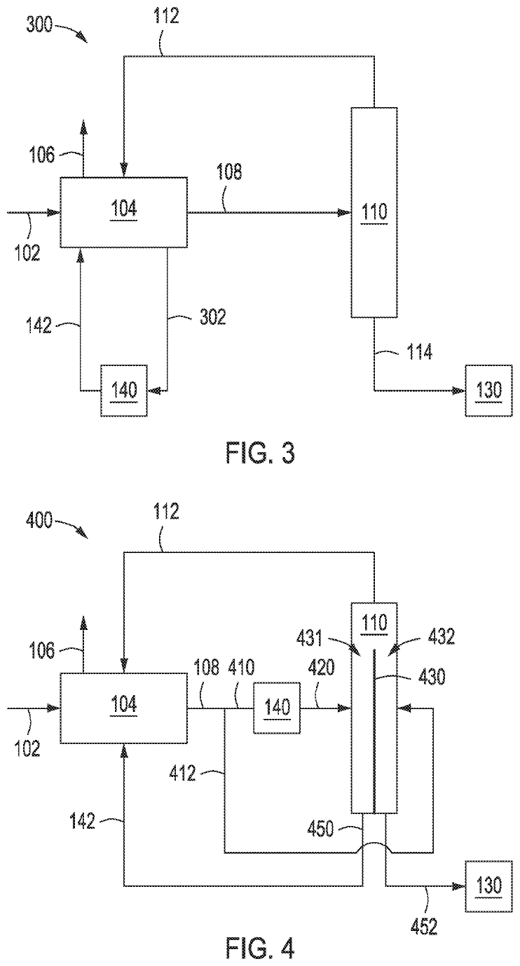

FIG. 3 is a block diagram of a system and process for xylene separation and liquid-phase isomerization according to a third embodiment of the present disclosure. In system and process 300, feed mixture 102 is received in p-xylene recovery unit 104 and is separated into p-xylene product stream 106 and raffinate stream 108 in a similar manner to that described above for system and process 100 in FIG. 1. Raffinate stream 108 is provided to distillation column 110 and separated into overhead stream 112 and lower stream 114. A majority of overhead stream 112 comprises toluene, and preferably, overhead stream 112 comprises at least a majority of the toluene present in raffinate stream 108, and/or preferably, overhead stream 112 consists essentially of toluene. Overhead stream 112 is fed to p-xylene recovery unit 104 as at least a portion of the desorbent used therein. Lower stream 114 is fed to vapor-phase isomerization unit 130, which conducts vapor-phase isomerization of xylene isomers under vapor-phase isomerization conditions in the presence of a vapor-phase isomerization catalyst, as discussed in more detail above in reference to system and process 100 in FIG. 1.

In system and process 300, intermediate stream 302 is obtained from p-xylene recovery unit 104 and is recirculated therein after undergoing liquid-phase isomerization. Intermediate stream 302 comprises p-xylene at a concentration higher than in raffinate stream 108 and lower than in p-xylene product stream 106. The p-xylene concentration in intermediate stream 302 may further be lower than the p-xylene concentration in feed mixture 102. As depicted in FIG. 3, intermediate stream 302 is fed to liquid-phase isomerization unit 140, which conducts liquid-phase isomerization of xylene isomers under liquid-phase isomerization conditions in the presence of a liquid-phase isomerization catalyst. Under the liquid-phase isomerization conditions in liquid-phase isomerization unit 140, additional p-xylene is produced from the xylene isomers to afford isomerized recycle stream 142 that is rich in p-xylene relative to intermediate stream 302. Isomerized recycle stream 142 is then fed to p-xylene recovery unit 104. As depicted in FIG. 3, isomerized recycle stream 142 is returned to p-xylene recovery unit 104 at a location different from that at which feed mixture 102 is provided to p-xylene recovery unit 104 it is to be appreciated, however, that at least a portion of isomerized recycle stream 142 may be combined with feed mixture 102 and/or returned to p-xylene recovery unit 104 at the same location where feed mixture 102 is introduced.

FIG. 4 is a block diagram of a system and process for xylene separation and liquid-phase isomerization according to a fourth embodiment of the present disclosure. In system and process 400, feed mixture 102 is received in p-xylene recovery unit 104 and separated into p-xylene product stream 106 and raffinate stream 108 in a similar manner to that described above for system and process 100 in FIG. 1.

In system and process 400, raffinate stream 108 is split into first stream 410 and second stream 412. First stream 410 is fed to liquid-phase isomerization unit 140, which conducts liquid-phase isomerization of xylene isomers under liquid-phase isomerization conditions in the presence of a liquid-phase isomerization catalyst. Under the liquid-phase isomerization conditions in liquid-phase isomerization unit 140, additional p-xylene is produced from the xylene isomers to afford isomerized raffinate stream 420, which is rich in p-xylene relative to the portion of raffinate stream 108 within first stream 410.

Isomerized raffinate stream 420 and second stream 412 (containing a non-isomerized portion of raffinate stream 412) are fed to distillation column 110 containing wall 430. Wall 430 extends upwardly from a bottom surface of distillation column 110 but does not reach a top surface thereof, thereby dividing distillation column 110 into first side 431 and second side 432 that are in vapor communication with one another. Isomerized raffinate stream 420 is fed to first side 431 of distillation column 110, and second stream 412 is fed to second side 432 of distillation column 110. Overhead stream 112 is obtained from distillation column 110, and a majority of overhead stream 112 comprises toluene. Overhead stream 112 represents a combined vapor stream received from first side 431 and second side 432 of distillation column 110. Preferably, overhead stream 112 comprises at least a majority of the toluene within isomerized raffinate stream 420 and the portion of raffinate stream 108 within second stream 412, and/or preferably, overhead stream 112 consists essentially of toluene. Overhead stream 112 is fed to p-xylene recovery unit 104 as at least a portion of the desorbent used therein.

At least two lower streams are obtained from distillation column 110. Specifically, first lower stream 450 is obtained from first side 431, and second lower stream 452 is obtained from second side 432. Since second lower stream 452 was produced directly from the portion of raffinate stream 108 within second stream 412, second lower stream 452 remains lean in p-xylene. Accordingly, second lower stream 452 is fed to vapor-phase isomerization unit 130, which conducts vapor-phase isomerization of xylene isomers under vapor-phase isomerization conditions in the presence of a vapor-phase isomerization catalyst, to convert one or more xylene isomers into additional p-xylene, as discussed in more detail above in reference to system and process 100 in FIG. 1.

Since first lower stream 450 was produced from isomerized raffinate stream 420 on first side 431 of distillation column 110, first lower stream 450 is rich in p-xylene relative to the portion of raffinate stream 108 within first stream 410. Accordingly, at least a portion of first lower stream 450 is fed to p-xylene recovery unit 104 as isomerized recycle stream 142 for additional p-xylene recovery therefrom. As depicted in FIG. 4, isomerized recycle stream 142 is returned to p-xylene recovery unit 104 at a location different from that at which feed mixture 102 is provided to p-xylene recovery unit 104. It is to be appreciated, however, that at least a portion of isomerized recycle stream 142 may be combined with feed mixture 102 and/or returned to p-xylene recovery unit 104 at the same location where feed mixture 102 is introduced.

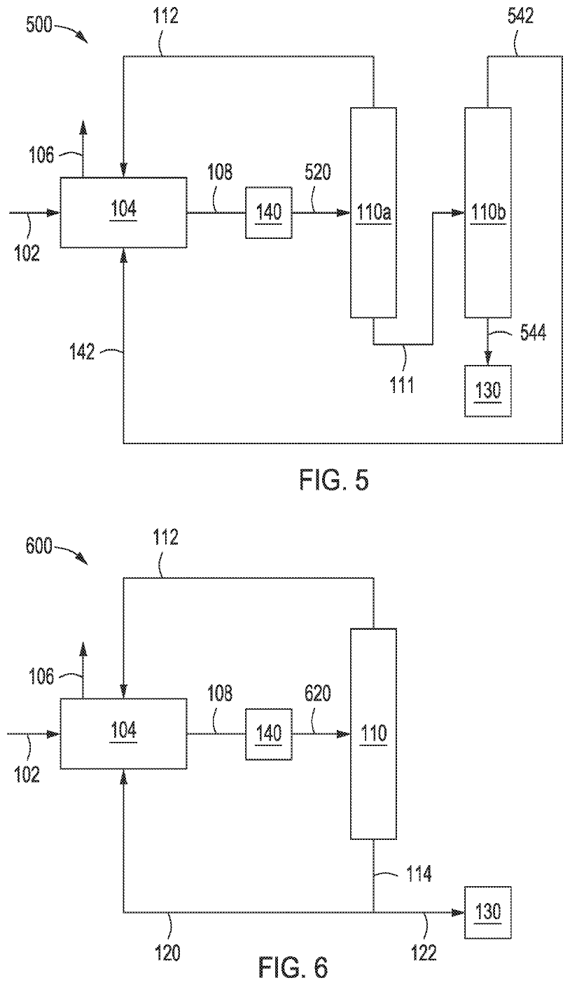

Distillation column 110 containing wall 430 (i.e., a divided wall column) is capable of separating multiple streams (e.g., overhead stream 112 and first lower stream 450) that may be fed directly to p-xylene recovery unit 104 for affecting further p-xylene separation. It is to be appreciated that multiple streams of a like nature may be similarly produced using two distillation columns in series with one another, but without splitting raffinate stream 108, as shown in FIG. 5.

FIG. 5 is a block diagram of a system and process for xylene separation and liquid-phase isomerization according to a fifth embodiment of the present disclosure. In system and process 500, feed mixture 102 is received in p-xylene recovery unit 104 and is separated into p-xylene product stream 106 and raffinate stream 108 in a similar manner to that described above for system and process 100 in FIG. 1. Raffinate stream 108 is fed to liquid-phase isomerization unit 140, which conducts liquid-phase isomerization of xylene isomers in the raffinate stream under liquid-phase isomerization conditions in the presence of a liquid-phase isomerization catalyst. Under the liquid-phase isomerization conditions in liquid-phase isomerization unit 140, additional p-xylene is produced from the xylene isomers to afford isomerized raffinate stream 520, which is rich in p-xylene relative to raffinate stream 108.

Isomerized raffinate stream 520 is fed to distillation column 110a, and overhead stream 112 is obtained, a majority of which comprises toluene. Preferably, overhead stream 112 comprises at least a majority of the toluene within isomerized raffinate stream 520, and/or preferably, overhead stream 112 consists essentially of toluene. Overhead stream 112 is fed to p-xylene recovery unit 104 as at least a portion of the desorbent used therein.

Lower stream 111 is also obtained from distillation column 110a and is fed to distillation column 110b. Lower stream 111 comprises xylene isomers and is rich in p-xylene relative to raffinate stream 108. Upon undergoing further separation in distillation column 110b, overhead stream 542 and lower stream 544 are obtained. Overhead stream 542 is rich in p-xylene relative to lower stream 111 and raffinate stream 108. At least a portion of overhead stream 542 is fed to p-xylene recovery unit 104 as isomerized recycle stream 142. As depicted in FIG. 5, isomerized recycle stream 142 is returned to p-xylene recovery unit 104 at a location different from that at which feed mixture 102 is provided to p-xylene recovery unit 104. It is to be appreciated, however, that at least a portion of isomerized recycle stream 142 may be combined with feed mixture 102 and/or returned to p-xylene recovery unit 104 at the same location where feed mixture 102 is introduced. Optionally, if excessive ethylbenzene is present in overhead stream 542, partial distillation may be performed to limit the introduction of ethylbenzene into overhead stream 542, in which case the amount of ethylbenzene and other xylene isomers present in lower stream 544 may be greater.

Lower stream 544 comprises C9+ aromatic hydrocarbons and possibly residual xylene isomers and may be fed to vapor-phase isomerization unit 130, which conducts vapor-phase isomerization of xylene isomers under vapor-phase isomerization conditions in the presence of a vapor-phase isomerization catalyst to convert one or more xylene isomers into additional p-xylene, as discussed in more detail above in reference to system and process 100 in FIG. 1. Alternately to vapor-phase isomerization, or in addition to vapor-phase isomerization, transalkylation may be conducted to convert C9+ aromatic hydrocarbons into additional xylene isomers. If performed, such transalkylation may preferably be performed prior to conducting vapor-phase isomerization in vapor-phase isomerization unit 130.

FIG. 6 is a block diagram of a system and process for xylene separation and liquid-phase isomerization according to a sixth embodiment of the present disclosure. In system and process 600, feed mixture 102, which comprises at least toluene, mixed xylenes and optionally ethylbenzene, is received in p-xylene recovery unit 104. Preferably, the amount of ethylbenzene is below a specified threshold amount and/or feed mixture 102 is processed to remove excess ethylbenzene therefrom, p-Xylene recovery unit 104 utilizes simulated moving bed chromatography with toluene as a desorbent to produce p-xylene product stream 106, which is rich in p-xylene and may consist essentially of p-xylene, and raffinate stream 108, which is lean in p-xylene. Raffinate stream 108 may be rich in at least one of o-xylene and m-xylene and contain toluene and optionally at least some ethylbenzene.

In system and process 600, raffinate stream 108 undergoes liquid-phase isomerization in liquid-phase isomerization unit 140. Under the liquid-phase isomerization conditions in liquid-phase isomerization unit 140, additional p-xylene is produced from the xylene isomers to afford isomerized raffinate stream 620, which is rich in p-xylene relative to raffinate stream 108.

Isomerized raffinate stream 620 is provided to distillation column 110 and separated into overhead stream 112 and lower stream 114. A majority of overhead stream 112 comprises toluene, and preferably, overhead stream 112 comprises at least a majority of the toluene within raffinate stream 108 and/or preferably, overhead stream consists 112 essentially of toluene. Overhead stream 112 is fed (recycled) to p-xylene recovery unit 104 as at least a portion of the desorbent used therein. Additional toluene desorbent may be fed to p-xylene recovery unit 104 from an external source (not shown in FIG. 6).

As depicted in FIG. 1, lower stream 114 is split into first stream 120 and second stream 122. However, splitting of lower stream 114 into first stream 120 and second stream 122 is optional, depending on factors such as, for example, if byproducts such as ethylbenzene have increased to levels prompting need for removal by vapor-phase isomerization or transalkylation. If produced, second stream 122 may be fed to vapor-phase isomerization unit 130, which conducts vapor-phase isomerization of xylene isomers under vapor-phase isomerization conditions in the presence of a vapor-phase isomerization catalyst, to convert one or more xylene isomers into additional p-xylene, as discussed in more detail above in reference to system and process 100 in FIG. 1.

First stream 120 is fed to p-xylene recovery unit 104 to affect separation of additional p-xylene in p-xylene product stream 106. As depicted in FIG. 1, first stream 120 is returned to p-xylene recovery unit 104 at a location different from that at which feed mixture 102 is introduced to p-xylene recovery unit 104. It is to be appreciated, however, that first stream 120 may be combined with feed mixture 102 and/or returned to p-xylene recovery unit 104 at the same location where feed mixture 102 is introduced.

Liquid-Phase Isomerization of C8+ Aromatic Hydrocarbons within a Feed Mixture

Liquid-phase isomerization may be desirable for producing p-xylene from various feed streams that are lean in p-xylene. Preferably, feed streams having low levels of ethylbenzene or feed streams that may be refined to afford levels of ethylbenzene below a specified threshold may be utilized in such liquid-phase isomerization processes, since ethylbenzene undergoes relatively slow isomerization under liquid-phase isomerization conditions and may otherwise gradually accumulate in an isomerized recycle stream produced under liquid-phase isomerization conditions. Moreover, the liquid-phase isomerization catalysts and liquid-phase isomerization conditions described herein further do not tend to produce significant quantities of ethylbenzene. Once removal of ethylbenzene or other byproducts become warranted, vapor-phase isomerization may be further utilized in combination with liquid-phase isomerization for further processing of a stream lean in p-xylene, since ethylbenzene and other byproducts that are not readily isomerized under liquid-phase isomerization conditions may undergo ready conversion to p-xylene and other value components under vapor-phase isomerization conditions. Such vapor-phase isomerization processes may occur continuously or on an as-needed basis. Through utilization of the liquid-phase isomerization and separation processes disclosed herein, less energetic separation of p-xylene from a feed mixture may be realized.

In the advantaged separation processes disclosed herein, simulated moving bed chromatography may be utilized to promote p-xylene separation from a feed mixture containing C7+ aromatic hydrocarbons and optionally C6+ aromatic hydrocarbons. Upon separation of p-xylene from a feed mixture containing toluene, a raffinate stream lean in p-xylene and further containing toluene may be produced. The toluene may be separated from the raffinate stream, either before or after conducting liquid-phase isomerization thereon, and provided as a desorbent for the simulated moving bed chromatography, thereby providing synergy for the combined isomerization and separation processes disclosed herein.

To support the foregoing, a liquid-phase isomerization catalyst having selectivity toward promoting isomerization of C8 aromatic hydrocarbons in preference to C7 aromatic hydrocarbons (toluene) and/or C9+ aromatic hydrocarbons may be used. Catalyst preference of this type allows isomerization of a raffinate stream lean in p-xylene or various streams derived therefrom to be isomerized under liquid-phase isomerization conditions to produce additional p-xvlene for separation (e.g. by simulated moving bed chromatography), even when significant quantities of toluene and/or C9+ aromatic hydrocarbons are present. A variety of process configurations for conducting the liquid-phase isomerization in accordance with the foregoing may be suitable, as discussed in more detail above in reference to FIGS. 1-6. For example, C8 aromatic hydrocarbons may be isomerized by liquid-phase isomerization prior to separating at least a portion of the toluene from at least a portion of the raffinate stream, and/or at least a portion of the toluene may be separated prior to conducting liquid-phase isomerization upon at least a portion of the raffinate stream. Moreover, although a liquid-phase isomerization catalyst having high selectivity toward isomerization of C8 aromatic hydrocarbons may be desirable, the separation and isomerization processes disclosed herein are sufficiently flexible to accommodate liquid-phase isomerization catalysts that are not completely selective for promoting isomerization of C8 aromatic compounds. As such, a range of suitable liquid-phase isomerization catalysts may also be accommodated in the processes disclosed herein. Additional details regarding suitable liquid-phase isomerization catalysts are provided below.

Accordingly, the present disclosure provides isomerization and separation processes comprising: (I) providing a feed mixture comprising one or more xylene isomers and optionally ethylbenzene; (II) separating the feed mixture in a p-xylene recovery unit using simulated moving bed chromatography with toluene as a desorbent to obtain a product stream rich in p-xylene, a raffinate stream lean in p-xylene, and optionally, an intermediate stream comprising p-xylene at a concentration higher than in the raffinate stream and lower than in the product stream, the raffinate stream comprising toluene, optionally ethylbenzene, and o-xylene, m-xylene, or any combination thereof; (III) optionally, conducting liquid-phase isomerization under liquid-phase isomerization conditions in the presence of a liquid-phase isomerization catalyst upon at least a portion of the raffinate stream to obtain an isomerized raffinate stream; (IV) separating at least a portion of the raffinate stream and/or, if present, at least a portion of the isomerized raffinate stream in a distillation column to obtain an overhead stream comprising at least a majority of the toluene in the raffinate stream and one or more lower streams each comprising one or more xylene isomers; (V) feeding at least a portion of the overhead stream to the p-xylene recovery unit as at least a portion of the desorbent; (VI) conducting liquid-phase isomerization under liquid-phase isomerization conditions in the presence of a liquid-phase isomerization catalyst and obtaining one or more isomerized recycle streams after conducting the liquid-phase isomerization, the liquid-phase isomerization being conducted upon at least one of: (a) at least a portion of the one or more lower streams; and/or (b) if present, at least a portion of the intermediate stream; and/or (c) the liquid-phase isomerization of at least a portion of the raffinate stream is carried out in (III); and
(VII) feeding at least a portion of the one or more isomerized recycle streams to the p-xylene recovery unit.

When produced by simulated moving bed chromatography, the product stream rich in p-xylene may comprise p-xylene at a concentration of ≥about 95%, ≥about 97%, ≥about 98%, ≥about 99%, or even ≥about 99.5%, based on the total mass.

Suitable feed mixtures for use in the disclosure herein may include, but are not limited to, those obtained from a catalytic reforming process, a benzene or toluene alkylation process, a xylene isomerization process, a toluene disproportionation process, a transalkylation process, cracking, a petroleum source, a bio-production source, or any combination thereof. In addition to one or more xylene isomers, the feed mixture may comprise ethylbenzene in an amount up to about 30 wt % or up to about 20 wt % of the total feed mixture. Preferably, the feed mixture may comprise ethylbenzene in an amount below a specified threshold amount or the feed stream mixture may be pre-processed/refined in a suitable manner to decrease the amount of ethylbenzene below the specified threshold amount. More preferably, the feed mixture may be produced or sourced with a low level of ethylbenzene, such that the feed mixture may be used directly without further refining the feed mixture to remove at least a portion of the ethylbenzene, which may be time-consuming, energy-intensive, and/or costly. Starting with a lower level of ethylbenzene may further reduce the burden or frequency of vapor-phase isomerization conducted herein. For example, suitable feed mixtures may preferably comprise ethylbenzene at about 2000 ppm or less, or about 1500 ppm or less, or about 1000 ppm or less based on total mass, or be further processed to afford an ethylbenzene concentration below these values. Particularly advantageous feed mixtures may be produced via toluene alkylation with methanol and/or dimethyl ether as an alkylation agent, which may afford p-xylene in considerably greater than equilibrium quantities relative to other xylene isomers, particularly o-xylene, as well as limit production of ethylbenzene (e.g., <2000 ppm by weight) and other problematic byproducts. Description of exemplary methylation catalysts, methylation agents, and methylation conditions for lower aromatic hydrocarbons may be found in, for example, U.S. Pat. No. 6,423,879; 6,504,072; 6,642.426, and 9,440,893, the relevant contents of which are incorporated herein by reference.

The feed mixture may comprise p-xylene and other C8 aromatic hydrocarbons at various concentrations of xylenes. The feed mixture may comprise an equilibrium or non-equilibrium distribution of xylene isomers. In non-limiting examples, a total concentration of xylene isomers (including ethylbenzene) may range from c(xylenes)1 to c(xylenes)2 t %, based on the total weight of the feed mixture, where c(xylenes)1 and c(xylenes)2 can be, independently, 30, 40, 50, 55, 60, 65, 70, 75, 80, 85, 90, 91, 92, 93, 94, 95, 96, 97, 98, 99, or even 100, as long as c(xylenes)1<c(xylenes)2. Preferably, c(xylenes)1 is 70 wt % or above. More preferably, c(xylenes)1 is 80 wt % or above. Even more preferably, the feed mixture may consist essentially of the xylene isomers.

In non-limiting examples, a total concentration of p-xylene in the feed mixture may range from c(pX)1 to c(pX)2 wt %, based on the total weight of the feed mixture, where c(pX)1 and c(pX)2 can be, independently, 10, 20, 30, 40, 50, 55, 60, 65, 70, 75, 80, 85, 90, 91, 92, 93, 94, 95, 96, 97, 98, 99, or even 100, as long as c(pX)1<c(pX)2. Preferably, c(pX)1 is 30 wt % or above. More preferably, c(pX)1 is 50 wt % or above.

The feed mixture may comprise ethylbenzene at various concentrations. In non-limiting examples, the feed mixture may comprise ethylbenzene at a concentration ranging from c(EB)1 to c(EB)2 wt %, based on the total weight of the feed mixture, where c(EB)1 and c(EB)2 can be, independently, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, as long as c(EB)1<c(EB)2. Preferably, c(EB)2 is 20 wt % or below. More preferably, c(EB)2 is 10 wt % or below. More preferably, c(EB)2 is 5 wt % or below. Still more preferably, c(EB)2 is 2 wt % or below, or 1 wt % or below, more preferably about 2000 ppm or below, or about 1500 ppm or below, or about 1000 ppm or below. Optionally, a feed mixture having a higher ethylbenzene content may be further processed to achieve an ethylbenzene concentration in the foregoing ranges. A recycle stream returned to the p-xylene separation unit may comprise ethylbenzene in any of the foregoing amounts, but preferably the amount of ethylbenzene returned is kept as small as possible.

While feed mixtures or recycle streams containing low amounts of ethylbenzene may be desirable for use in the disclosure herein, it is to be understood that ethylbenzene and other byproducts may be effectively addressed in the disclosure herein through vapor-phase isomerization. Additional details regarding suitable vapor-phase isomerization catalysts and vapor-phase isomerization conditions are provided further below.

The feed mixture may comprise benzene, toluene, and C9+ hydrocarbons at various quantities. In non-limiting examples, the feed mixture may comprise benzene and toluene combined in a range from c(BT)1 to c(BT)2 wt %, based on the total weight of the feed mixture, where c(BT)1 and c(BT)2 can be, independently, 0.01, 0.1, 1.0, 2.0, 3.0, 5.0, 8.0, 10.0, 15.0, 20.0, 30.0, 40.0, or 50.0, as long as c(BT)1<c(BT)2. Preferably, c(BT)2 is 10.0 or less. More preferably, c(BT)2 is 5.0 or less. Still more preferably, c(BT)2 is 3.0 or less. In various embodiments, toluene may be the primary component between benzene and toluene, and in some embodiments, combined benzene and toluene may consist essentially of toluene. That is, in some embodiments, the feed mixture may be substantially free of benzene. In some or other non-limiting examples, the feed mixture may comprise C9+ hydrocarbons, in total, in a range from c(C9+)1 to c(C9+)2 wt %, based on the total weight of the feed mixture, where c(C9+)1 and c(C9+)2 can be, independently, 0.01, 0.1, 1.0, 5.0, 10.0, 20.0, as long as c(C9+)1<c(C9+)2.

The raffinate stream or one or more streams derived therefrom may contain a non-equilibrium distribution of xylene isomers and/or a lower concentration of p-xylene than is present in the feed mixture. In non-limiting examples, the total quantity of p-xylene in the raffinate stream or one or more streams derived therefrom may have a p-xylene concentration, based on mass of total xylene isomers in the raffinate stream, ranging from c(pX)1 to c(pX)2 wt %, where c(pX)1 and c(pX)2 can be, independently, 0, 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30, as long as c(pX)1<c(pX)2. Preferably, c(pX)2 is 20 or less, 15 or less, 12 or less, 10 or less, 8 or less, 6 or less, 5 or less, 4 or less, 2 or less, or 1 or less. In non-limiting examples, the total quantity of m-xylene in the raffinate stream or one or more streams derived therefrom may have a m-xylene concentration, based on mass of total xylene isomers in the raffinate stream, ranging from c(mX)1 to c(mX)2 wt %, where c(mX)1 and c(mX)2 can be, independently, 10, 15, 20, 25, 30, 35, 40, 45, 50, 55, 60, 65, 70, 75, 80, 85, 90, 95, as long as c(mX)1<c(mX)2. Preferably, c(mX)1 is 30 or greater and/or c(mX)2 is 80 or less. Preferably, c(mX)1 is 40 or greater and/or c(mX)2 is 80 or less. In non-limiting examples, the total quantity of o-xylene in the raffinate stream or one or more streams derived therefrom may have an o-xylene concentration, based on mass of total xylene isomers in the raffinate stream, ranging from c(oX)1 to c(oX)2 wt %, where c(oX)1 and c(oX)2 can be, independently, 10, 15, 20, 25, 30, 35, 40, 45, 50, 55, 60, 65, 70, 75, 80, 85, 90, 95, as long as c(oX)1<c(oX)2. Preferably, c(oX)1 is 10 or greater and/or c(oX)2 is 80 or less. Preferably, c(oX)1 is 10 or greater and/or c(oX)2 is 60 or less. Preferably, c(oX)1 is 10 or greater and/or c(oX)2 is 50 or less.

The one or more isomerized recycle streams fed to the p-xylene recovery unit may contain an equilibrium distribution of xylene isomers and/or a higher concentration of p-xylene than is present in the raffinate stream. In non-limiting examples, the total quantity of p-xylene in the one or more isomerized recycle streams may have a p-xylene concentration, based on mass of total xylene isomers in the one or more isomerized recycle streams, ranging from c(pX)1 to c(pX)2 wt %, where c(pX)1 and c(pX)2 can be, independently, 0, 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30, as long as c(pX)1<c(pX)2. Preferably, c(pX)2 is 20 or less, 15 or less, 12 or less, 10 or less, 8 or less, 6 or less, 5 or less, 4 or less, 2 or less, or 1 or less. In non-limiting examples, the total quantity of m-xylene in the one or more isomerized recycle streams may have a m-xylene concentration, based on mass of total xylene isomers in the one or more isomerized recycle streams, ranging from c(mX)1 to c(mX)2 wt %, where c(mX)1 and c(mX)2 can be, independently, 0, 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30, as long as c(mX)1<c(mX)2. Preferably, c(mX)2 is 20 or less, 15 or less, 12 or less, 10 or less, 8 or less, 6 or less, 5 or less, 4 or less, 2 or less, or 1 or less. In non-limiting examples, the total quantity of o-xylene in the one or more isomerized recycle streams may have a o-xylene concentration, based on mass of total xylene isomers in the one or more isomerized recycle streams, ranging from c(oX)1 to c(oX)2 wt %, where c(oX)1 and c(oX)2 can be, independently, 0, 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30, as long as c(oX)1<c(oX)2. Preferably, c(oX)2 is 20 or less, 15 or less, 12 or less, 10 or less, 8 or less, 6 or less, 5 or less, 4 or less, 2 or less, or 1 or less.

In various embodiments of the processes disclosed herein, 80 wt % or greater, preferably 85 wt % or greater, more preferably 90 wt % or greater, more preferably 95 wt % or greater, more preferably 98 wt % or greater, more preferably 99 wt % or greater, or still more preferably approximately 100 wt % of the feed mixture may be in liquid phase at the inlet of an isomerization reaction in which the liquid-phase isomerization takes place. The feed mixture may have an inlet temperature in the range from T1 to T2° C., where T1 and T2 can be, independently, 200, 210, 220, 230, 240, 250, 255, 260, 265, 270, 275, 280, 285, 290, 295, or 300, as long as T1<T2. The relatively low inlet temperature of the feed mixture, in combination with other liquid-phase isomerization conditions described below may facilitate the liquid-phase isomerization of C8 aromatic hydrocarbons to p-xylene for recycling to the p-xylene recovery unit.

Liquid-phase isomerization in the present disclosure may be conducted using a fixed bed reactor, a fluidized bed reactor, or a moving bed reactor. The feed provided to the liquid-phase isomerization conditions may be lean in p-xylene, such as a raffinate stream obtained following separation of p-xylene from a C8-containing feed mixture, an intermediate stream, or one or more lower streams following separation of raffinate in a distillation column. The feed provided to the liquid-phase isomerization conditions may flow upward, downward, or in a radial fashion within an isomerization reactor. Alternately, liquid-phase isomerization may be conducted batchwise in some instances.

Suitable liquid-phase isomerization conditions may include a reaction gauge pressure in an isomerization reactor ranging from p1 to p2 kPa, where p1 and p2 can be, independently, 1700, 1800, 1900, 2000, 2100, 2200, 2300, 2400, 2500, 2600, 2700, 2800, 2900, 3000, 3100, 3200, 3300, 3400, or 3500, as long as p1<p2. Preferably, p2 is 3000 kPa or lower. Preferably, p2 is 2500 kPa or lower. Higher reaction gauge pressures may promote dissolution of molecular hydrogen in the liquid phase in the isomerization reaction, wherein the molecular hydrogen is provided as a co-feed in combination with the feed mixture to promote the liquid-phase isomerization reaction.

Suitable liquid-phase isomerization conditions may include a reaction temperature ranging from T1 to T2° C., where T1 and T2 can be, independently 200, 210, 220, 230, 240, 250, 255, 260, 265, 270, 275, 280, 285, 290, 295, or 300, as long as T1<T2. The relatively low reaction temperature during liquid-phase isomerization may improve energy efficiency by requiring less energy to heat the feed undergoing isomerization and by not requiring condensation of large quantities of a high-temperature vapor-phase following vapor-phase isomerization.

Suitable liquid-phase isomerization conditions may include a high WHSV ranging from w1 to w2 hour⁻¹, where w1 and w2 can be, 5.0, 5.5, 6.0, 6.5, 7.0, 7.5, 8.0, 8.5, 9.0, 9.5, 10, 11, 12, 12.5, 13, 14, 15, 16, 17, 17.5, 18, 19, or 20, as long as w1<w2. High WHSV values may be facilitated by co-feeding molecular hydrogen at a suitable rate.

Molecular hydrogen may be optionally provided as a co-feed to the liquid-phase isomerization conditions. In certain embodiments, the molecular hydrogen co-fed into an isomerization reactor, or a portion thereof, can be introduced as a pressurized gas via an inlet upon the isomerization reactor. Additionally or alternatively, the molecular hydrogen or a portion thereof can be fed into a feeding line, a vessel, or a storage tank associated with a feed provided to the liquid-phase isomerization conditions, which may promote admixture of the molecular hydrogen with the feed and deliver the molecular hydrogen to the liquid-phase isomerization conditions in combination with the feed. A majority (for example, ≥50%, ≥60%, ≥70%, ≥80%, ≥90%, ≥95%, ≥98%), more preferably substantially the entirety (≥99%), of the molecular hydrogen may be dissolved in the liquid phase under the liquid-phase isomerization conditions. To achieve a higher concentration of dissolved molecular hydrogen in the liquid phase, a suitably high pressure may be maintained in the isomerization reactor.

In non-limiting examples, the molecular hydrogen can be fed into the isomerization reactor at a feeding rate of r(H2)1 to r(H2)2 ppm by weight, based on the total weight of the feed, where r(H2)1 and r(H2)2 can be, independently, 100, 150, 200, 250, 300, 350, 400, 450, 500, 550, 600, 650, 700, 750, 800, 850, 950, 1000, 1500, 2000, 2500, 3000, 3500, 4000, 4500, or 5000, as long as r(H2)1<r(H2)2. Preferably, r(H2)2 is 3000 or less, 2000 or less, 1000 or less, 800 or less, 600 or less, or 500 or less.

Suitable liquid-phase isomerization catalysts may comprise a zeolite having an MEL framework structure (e.g., ZSM-11), an MFI framework structure (e.g., ZSM-5), or any combination thereof. Other suitable examples of zeolites that may be effective for conducting liquid-phase isomerization may include, for example, those having a MWW framework, a MOR framework, or the like. Examples may include: MWW-22, MWW-49, MWW-54, and combinations thereof.

In certain embodiments, the liquid-phase isomerization catalyst may comprise a first metal element selected from Fe. Co, Ni, Ru, Rh, Pd, Re, Os, Ir, Pt, and combinations thereof, and optionally a second metal selected from Sn, Zn, Ag, and combinations thereof. The first metal element may catalyze hydrogenation of olefins that ma be produced under the liquid-phase isomerization, such as those produced by dealkylation of ethylbenzene. The second metal element may promote or enhance the catalytic effect of the first metal element. In other embodiments, the liquid-phase isomerization catalyst may be free of precious metal (i.e., Ru, Rh, Pd, Os, Ir, and Pt). In other embodiments, the liquid-phase isomerization catalyst may be free of any Group 7-10 metal. In still other embodiments, the liquid-phase isomerization catalyst may be free of any Group 7-15 metals except aluminum.

Zeolites having a MFI framework (e.g., ZSM-5) suitable for use in the present disclosure may have one or more of the following characteristics: presence in a hydrogen form (HZSM-5); a crystal size ≤0.1 micron; a mesoporous surface area (MSA) ≥45 m²/g; a total surface area to mesoporous surface area ratio 9; and a silica to alumina molar ratio in the range of 20 to 50.

Suitable zeolites having a MEL framework (e.g., ZSM-11) may comprise a plurality of primary crystallites, in which at least 75% (e.g., ≥80%, ≥85%, ≥90%, or even ≥95%) of the crystallites have crystallite size of less than or equal to 200 nanometer (e.g., ≤150, ≤100. ≤80, ≤50, ≤30 nanometers). Thus, at least 75% (e.g., ≥80%. ≥85%, ≥90%, or even ≥95%) of the crystallites may have a crystallite size in a range of cs1 to cs2 nanometers (nm), where cs1 and cs2 can be, independently, 5, 10, 20, 30, 40, 50, 60, 70, 80, 90, 100, 120, 140, 150, 160, 180, or 200, as long as cs1<cs2. Preferably, cs1 is 10 or more and cs2 is 150 or less. More preferably, cs1 is 10 or more and cs2 is 50 or less. In this disclosure, crystallite size may be defined as the largest dimension of the crystallite observed under a transmission electron microscope (“TEM”). To determine crystallite size, a sample of the zeolite material is placed in a TEM, and an image of the sample is taken. The image is then analyzed to determine the crystallite size and distributions thereof. The small crystallite sizes of the MEL framework type zeolite material of this disclosure gives rise to surprisingly high catalytic activities and other advantages, in addition to the surprising tolerance toward toluene reactivity under liquid-phase isomerization conditions.

Optionally, the primary crystallites of zeolites having a MEL framework may have an average primary crystallite size of less than 80 nm, preferably less than 70 nm, and in some cases less than 60 nm, in each of the a, b and c crystal vectors as measured by X-ray diffraction. The primary crystallites may optionally have an average primary crystallite size of greater than 20 nm, optionally greater than 30 nm, in each of the a, b and c crystal vectors, as measured by X-ray diffraction.

The primary crystallites may have a narrow particle size distribution such that at least 90% of the primary crystallites by number have a primary crystallite size in the range of 10 to 80 nm, preferably in the range of from 20 to 50 nm, as determined by analysis of images of the primary crystallites taken by TEM.

Crystallites of zeolites having a MEL framework may assume various shapes such as substantially spherical, rod-like, or the like. Alternately or in addition, the crystallites can have irregular shapes in TEM images. Thus, a crystallite may exhibit a longest dimension in a first direction (“primary dimension”), and a width in another direction perpendicular to the first direction (“secondary dimension”), where the width is defined as the dimension in the middle of the primary dimension, as determined by TEM image analysis. The ratio of the primary dimension to the width is called the aspect ratio of the crystallite. In certain embodiments, the crystallites can have an average aspect ratio determined by TEM image analysis in a range from ar1 to ar2, where ar1 and ar2 can be, independently, 1, 1.2, 1.4, 1.5, 1.6, 1.8, 2.0, 2.2, 2.4, 2.5, 2.6, 2.8, 3.0, 3.2, 3.4, 3.5, 3.6, 3.8, 4.0, 4.2, 4.4, 4.5, 4.6, 4.7, 4.8, or 5.0, as long as ar1<ar2. Preferably, ar1 is 1 or greater and ar2 is 3 or less, or ar1 is 1 or greater and ar2 is 2 or less.

The small crystallites of zeolites having a MEL framework may aggregate to form agglomerates. The agglomerates are polycrystalline materials having void space at the boundary of the crystallites. The agglomerates may be formed from primary crystallites having an average primary crystallite size as determined by TEM image analysis of less than 80 nm, preferably less than 70 nm and more preferably less than 60 nm, or even less than 50 nm.

Suitable zeolites having a MEL framework may comprise a mixture of agglomerates of the primary crystallites together with some unagglomerated primary crystallites. The majority of the zeolites having a MEL framework may comprise, for example, greater than 50 wt % or greater than 80 wt % may comprise agglomerates of primary crystallites. The agglomerates can be regular or irregular form. For more information on agglomerates please see Walter, D. (2013) Primary Particles-Agglomerates-Aggregates, in Nanomaterials (ed Deutsche Forschungsgemeinschaft (DFG)), Wiley-VCH Verlag GmbH & Co. KGaA, Weinheim, Germany, doi; 10.1002/9783527673919, pages 1-24.

Preferably, zeolites having a MEL framework may comprise less than 10% by weight of primary crystallites having a size of >200 nm as determined by TEM image analysis, or less than 10% by weight of primary crystallites having a size of >150 nm as determined by TEM image analysis, or less than 10% by weight of primary crystallites having a size of >100 nm as determined by TEM image analysis, or less than 10% by weight of primary crystallites having a size of >80 nm as determined by TEM image analysis.

Suitable zeolites having a MEL framework may have a silica to alumina ratio of R(s/a) that can vary from r1 to r2, where r1 and r2 can be, independently, 10, 12, 14, 15, 16, 18, 20, 22, 24, 25, 26, 28, 30, 32, 34, 35, 36, 38, 40, 42, 44, 45, 46, 48, 50, 52, 54, 55, 56, 58, or 60, as long as r1<r2. Preferably, r1 is 20 or greater and r2 is 50 or less. Preferably, r1 is 20 or greater and r2 is 40 or less. Preferably, r1 is 20 or greater and r2 is 30 or less. Ratio R(s/a) can be determined by ICP-MS (inductively coupled plasma mass spectrometry) or XRF (X-ray fluorescence).

Suitable zeolites having a MEL framework may have a BET total specific surface area of A(st) that can vary from a1 to a2 m²/g, where a1 and a2 can be, independently, 300, 320, 340, 350, 360, 380, 400, 420, 440, 450, 460, 480, 500, 520, 540, 550, 560, 580, or 600, as long as a1<a2. Preferably, a1 is 400 or greater and a2 is 500 or less. Preferably, a1 is 400 or greater and a2 is 475 or less. A(st) can be determined by the BET method (Brunauer-Emmet-Teller method, a nitrogen adsorption method). The high total surface area A(st) of the zeolite material of this disclosure is another reason why it exhibits high catalytic activity for converting aromatic hydrocarbons. The BET method can yield a total specific area of a measured material, including a microporous specific area component and a mesopore specific area component. The mesopore specific area may be called mesopore area, mesoporous area, or external area in this disclosure. The total specific area may be called total surface area or total area in this disclosure.

Suitable zeolites having a MEL framework may have a mesopore area of A(mp) that is ≥15% (e.g., ≥16%, ≥18%, ≥20%, ≥22%. ≥24%, ≥25%) of the total surface area A(st) discussed above. In certain embodiments it is preferred that A(mp) ≥20%*A(st). In certain embodiments, it is preferred that A(mp)≤40%*A(st). In certain embodiments, it is preferred that A(mp)≤30%*A(st). The high mesopore area A(mp) of the zeolite material of this disclosure is another reason why it exhibits a high catalytic activity for converting aromatic hydrocarbons. Without intending to be bound by a particular theory, it is believed that the catalytic sites present on the mesopore area of the zeolite material of this disclosure are more numerous due to the high mesopore area, which tend to contribute more to the catalytic activity than are catalytic sites located in deep channels inside the zeolite material. The time required for reactant molecules to reach the catalytic sites on the mesopore surfaces and the product molecules to exit them is relatively short. Conversely, it would take significantly longer time for reactant molecules to diffuse into deep channels and for the product molecules to diffuse out of them.

Suitable zeolites having a MEL framework may have a hexane sorption value of v(hs) that can vary from v1 to v2 mg/g, where v1 and v2 can be, independently, 90, 92, 94, 95, 96, 98, 100, 102, 104, 105, 106, 108, or 110, as long as v1<v2. Hexane sorption value can be determined by TGA (thermogravimetric analysis) as is typical in the industry.

Suitable zeolites having a MEL framework may have an alpha value that can vary from a1 to a2, where a1 and a2 can be, independently, 500, 600, 700, 800, 900, 1000, 1200, 1400, 1500, 1600, 1800, 2000, 2200, 2400, 2500, 2600, 2800, or 3000, as long as a1<a2. Alpha value can be determined by the method described in U.S. Pat. No. 3,354,078 and Journal of Catalysis, Vol. 4, p. 527 (1965); vol. 6, p. 278 (1966) and Vol. 61, p. 395 (1980).

Optionally, suitable zeolites having a MEL framework may be calcined and subjected to post-treatments such as steaming and/or acid washing. Steaming may be conducted at a temperature of at least 200° C., preferably at least 350° C., more preferably at least 400° C., in some cases at least 500° C., for a period of from 1 to 20 hours, preferably from 2 to 10 hours. Acid washing may be conducted with an aqueous solution of an acid, preferably an organic acid, such as a carboxylic acid, preferably oxalic acid. Optionally, a steamed zeolite may be treated with an aqueous solution of an acid at a temperature of at least 50° C., preferably at least 60° C., for a period of at least 1 hour, preferably at least 4 hours, for example, in the range of from 5 to 20 hours. Preferably, a treated zeolite having a MEL framework may have a chemical composition with a molar ratio of nSiO₂:Al₂O₃, wherein n is at least 20, more preferably at least 50, and in some cases at least 100.

The liquid-phase isomerization catalysts suitable for use in the disclosure herein may be formulated with a binder or present as an unbound free powder. The binder may comprise a binder material resistant to the temperature and other liquid-phase isomerization conditions. Examples of suitable binder materials include clays, alumina, silica, silica-alumina, silica-magnesia, silica-zirconia, silica-thoria, silica-beryllia, and silica-titania, as well as ternary compositions, such as silica-alumina-thoria, silica-alumina-zirconia, silica-alumina-magnesia and silica-magnesia-zirconia. In non-limiting examples, a binder material may be included with the liquid-phase isomerization catalyst at a concentration from c1 to c2 wt %, based on the total weight of the catalyst, where c1 and c2 can be, independently, 1, 5, 10, 15, 20, 25, 30, 35, 40, 45, 50, 55, 60, 65, 70, 75, 80, 85, 90, 95, 98, 99, as long as c1<c2. The inclusion of a binder in the isomerization catalyst can enhance its mechanical strength, among other factors. A zeolite capable of promoting liquid-phase isomerization may also be blended with a second zeolite as a binder material, thereby forming a zeolite-bound zeolite, as described in U.S. Pat. Nos. 5,993,642 and 5,994,603 and each incorporated herein by reference. The relative proportions of zeolite and binder material may range from about 1:99 to about 99:1 on a mass basis. In illustrative examples, the zeolite capable of promoting liquid-phase isomerization may be present in an amount of 10% to about 70% by mass of the zeolite-bound zeolite, or about 20% to about 50% by mass of the zeolite-bound zeolite.

The liquid-phase isomerization catalyst may be a freshly made catalyst, a regenerated catalyst, or a mixture thereof. Regeneration of the catalyst may be conducted in the isomerization reactor after the catalyst activity has decreased to a threshold level at the end of catalyst cycle, such as by exposing the catalyst to a stream of gas comprising molecular hydrogen. Alternatively, ex situ regeneration of the catalyst may be implemented, where the spent catalyst is taken out of the isomerization reactor, heated in an oxygen-rich environment and/or exposed to a gas stream comprising molecular hydrogen to abate coke on its surface.

As discussed above, the present disclosure may accommodate liquid-phase isomerization of various streams to convert a stream lean in p-xylene into a recycle stream enriched in p-xylene for separation in a p-xylene recovery unit. In non-limiting examples, at least a portion of a raffinate stream, at least a portion of an intermediate stream, or at least a portion of a lower stream obtained from distillation may undergo liquid-phase isomerization in the disclosure herein. The raffinate stream and/or the intermediate stream (when produced) may comprise up to about 50 vol % toluene. Similarly, the one or more isomerized recycle streams may comprise up to about 50 vol % toluene, depending on the amount of toluene present in a stream from which the isomerized recycle stream(s) were produced.

In some embodiments, liquid-phase isomerization in (III) above may be carried out upon at least a portion of the raffinate stream to obtain an isomerized raffinate stream, conducted in (III) above upon. In some embodiments, at least a portion of the isomerized raffinate stream may be fed to the distillation column providing the overhead stream and one or more lower streams. In other embodiments, at least a portion of the isomerized raffinate stream is not fed to the distillation column, in which case at least a portion of the isomerized raffinate stream may be fed to the p-xylene recovery unit as at least a portion of one or more isomerized recycle streams.

In some embodiments, the isomerized raffinate stream may be fed to the distillation column in (IV), and one or more lower streams may be obtained as at least a portion of one or more lower streams produced upon separating the isomerized raffinate stream in the distillation column. Preferably, the distillation column may comprise a divided wall distillation column, and a first lower stream rich in p-xylene may be obtained from a first side of the divided wall distillation column and a second lower stream lean in p-xylene may be obtained from a second side of the divided wall distillation column. The one or more isomerized recycle streams may be obtained as at least a portion of the first lower stream. When a divided wall distillation column is used, processes of the present disclosure may comprise feeding the isomerized raffinate stream to the first side of the divided wall distillation column, and feeding at least a portion of the raffinate stream to the second side of the divided wall distillation column. As an alternative to a divided wall distillation column, the distillation column may comprise a first distillation column and a second distillation column linked in series, in which the isomerized raffinate stream is fed to the first distillation column. In such a two-column approach, the overhead stream comprising at least a majority of the toluene may be obtained from the first distillation column, a first lower stream may be obtained from the first distillation column and fed to the second distillation column, an overhead stream rich in p-xylene may be obtained from the second distillation column and fed to the p-xylene recovery unit as at least a portion of the one or more isomerized recycle streams, and a second lower stream lean in p-xylene may be obtained from the second distillation column.

In some embodiments, the liquid-phase isomerization in (VI) may be carried out on upon at least a portion of the one or more lower streams to produce the one or more isomerized recycle streams. When liquid-phase isomerization is conducted upon the one or more lower streams, liquid-phase isomerization of the raffinate stream in (111) may not be carried out and/or an intermediate stream in (II) may not be produced. Preferably, the distillation column may produce one lower stream when liquid-phase isomerization of the one or more lower streams is conducted.

In some embodiments, an intermediate stream is obtained in (II), the liquid-phase isomerization is carried out upon at least a portion of the intermediate stream in (VI), and at least a portion of the one or more isomerized recycle streams in (VII) is obtained from the intermediate stream following liquid-phase isomerization thereon. Preferably, when an intermediate stream is produced, liquid-phase isomerization is not conducted upon the raffinate stream or one or more lower streams obtained from the distillation column. When produced, the intermediate stream may comprise up to about 50 vol % toluene.

Accordingly, in various embodiments, the liquid-phase isomerization in the disclosure herein may be conducted upon one of the intermediate stream, the raffinate stream, or the one or more lower streams.

In more specific embodiments, processes of the present disclosure utilizing liquid-phase isomerization of a portion of the raffinate stream may comprise: (i) providing a feed mixture comprising one or more xylene isomers and optionally ethylbenzene;
(ii) separating the feed mixture in a p-xylene recovery unit using simulated moving bed chromatography with toluene as a desorbent to obtain a product stream rich in p-xylene and a raffinate stream lean in p-xylene, the raffinate stream comprising toluene, optionally ethylbenzene, and o-xylene, m-xylene, or any combination thereof:
(iii) conducting liquid-phase isomerization under liquid-phase isomerization conditions in the presence of a liquid-phase isomerization catalyst upon at least a portion of the raffinate stream to obtain an isomerized raffinate stream; (iv) feeding the isomerized raffinate stream to a first side of a divided wall distillation column (i.e., a vessel having a first side and a second side divided by an intermediate wall attached to a bottom surface but not a top surface thereof); (v) feeding a portion of the raffinate stream to a second side of the divided wall distillation column; (vi) obtaining a first lower stream rich in p-xylene from the first side of the divided wall distillation column, a second lower stream lean in p-xylene from the second side of the divided wall distillation column, and an overhead stream rich in toluene from the divided wall distillation column; (vii) feeding at least a portion of the overhead stream to the p-xylene recovery unit as at least a portion of the desorbent; and (viii) feeding one or more isomerized recycle streams to the p-xylene recovery unit, the one or more isomerized recycle streams being obtained as at least a portion of the first lower stream.

In other more specific embodiments, processes of the present disclosure utilizing liquid-phase isomerization of one or more lower streams, an intermediate stream, or a portion of the raffinate stream may comprise: (A) providing a feed mixture comprising one or more xylene isomers and optionally ethylbenzene; (B) separating the feed mixture in a p-xylene recovery unit using simulated moving bed chromatography with toluene as a desorbent to obtain a product stream rich in p-xylene, a raffinate stream lean in p-xylene, and optionally an intermediate stream comprising p-xylene at a concentration higher than in the raffinate stream and lower than in the product stream, the raffinate stream comprising toluene, optionally ethylbenzene, and o-xylene, m-xylene, or any combination thereof; (C) separating at least a portion of the raffinate stream in a distillation column to obtain an overhead stream comprising at least a majority of the toluene in the raffinate stream and one or more lower streams each comprising one or more xylene isomers; (D) feeding at least a portion of the overhead stream to the p-xylene recovery unit as at least a portion of the desorbent; (E) conducting liquid-phase isomerization under liquid-phase isomerization conditions in the presence of a liquid-phase isomerization catalyst to produce one or more isomerized recycle streams, the liquid-phase isomerization being conducted upon at least one of: (a) at least a portion of the one or more lower streams; and/or (b) if present, at least a portion of the intermediate stream; and/or (c) at least a portion of the raffinate stream; and (F) feeding at least a portion of the one or more isomerized recycle streams to the p-xylene recovery unit.

In one or more embodiments, the liquid-phase isomerization may be conducted upon at least a portion of the one or more lower streams, and at least a portion of the one or more isomerized recycle streams may be obtained from the one or more lower streams after conducting the liquid-phase isomerization thereon.

In one or more embodiments, the liquid-phase isomerization may be conducted upon at least a portion of the raffinate stream to produce an isomerized raffinate stream, and at least a portion of the one or more isomerized recycle streams may be obtained from the isomerized raffinate stream.

In one or more embodiments, the intermediate stream may be obtained, the liquid-phase isomerization may be conducted upon at least a portion of the intermediate stream, and at least a portion of the one or more isomerized recycle streams may be obtained from the intermediate stream after conducting the liquid-phase isomerization thereon.

### Vapor-Phase Isomerization

In any of the foregoing embodiments, a lower stream lean in p-xylene may be further processed by vapor-phase isomerization, such as to isomerize ethylbenzene or other byproducts present therein. Vapor-phase isomerization may be favored over liquid-phase isomerization when ethylbenzene is present at a high concentration, such as when ethylbenzene is present in an amount greater than or equal to about 10 wt %, based on the total mass. Such vapor-phase isomerization may be conducted under vapor-phase isomerization conditions in the presence of a suitable vapor-phase isomerization catalyst, as described in further detail hereinafter.

Suitable vapor-phase isomerization conditions may include a temperature and a pressure such that a majority of the xylenes are in a vapor-phase. Description of exemplary vapor-phase isomerization processes, conditions, and catalysts can be found in, for example, U.S. Patent Application Publications 2011/03196881; 2012/0108867; 2012/0108868; 2014/0023563; 2015/0051430; and 2017/0081259, the relevant contents of which are incorporated herein by reference.

In one example, suitable vapor-phase isomerization catalyst may include zeolites having a MWW framework. Such zeolites may have a Constraint Index <5 and include molecular sieves having one or more of the following properties:
a) molecular sieves made from a common first degree crystalline building block unit cell, which unit cell has the MWW framework topology. (A unit cell is a spatial arrangement of atoms which if tiled in three-dimensional space describes the crystal structure. Such crystal structures are discussed in the “Atlas of Zeolite Framework Types”. Fifth edition, 2001, incorporated herein by reference);
b) molecular sieves made from a second degree building block, being a 2-dimensional tiling of such MWW framework topology unit cells, forming a monolayer of one unit cell thickness, in an embodiment, one c-unit cell thickness;
c) molecular sieves made from common second degree building blocks, being layers of one or more than one unit cell thickness, where the layer of more than one unit cell thickness is made from stacking, packing, or binding at least two monolayers of MWW framework topology unit cells. The stacking of such second degree building blocks can be in a regular fashion, an irregular fashion, a random fashion, or any combination thereof; and
d) molecular sieves made by any regular or random 2-dimensional or 3-dimensional combination of unit cells having the MWW framework topology.

Example zeolites having a MWW framework include MCM-22 (U.S. Pat. No. 4,954,325), PSH-3 (U.S. Pat. No. 4,439,409). SSZ-25 (U.S. Pat. No. 4,826,667), ERB-1 (European Patent No. 0293032), ITQ-1 (U.S. Pat. No. 6,077,498), ITQ-2 (International Publication No. WO97/17290), MCM-36 (U.S. Pat. No. 5,250,277), MCM-49 (U.S. Pat. No. 5,236,575). MCM-56 (U.S. Pat. No. 5,362,697). UZM-8 (U.S. Pat. No. 6,756,030). UZM-8HS (U.S. Pat. No. 7,713,513). UZM-37 (U.S. Pat. No. 7,982,084). EMM-10 (U.S. Pat. No. 7,842,277). EMM-12 (U.S. Pat. No. 8,704,025). EMM-13 (U.S. Pat. No. 8,704,023), UCB-3 (U.S. Pat. No. 9,790,143B2) and mixtures thereof.

In some embodiments, the zeolites having a MWW framework may be contaminated with other crystalline materials, such as ferrierite or quartz, which may be present in quantities of 10 wt % or <5 wt %.

The present disclosure can further include the following non-limiting aspects and/or embodiments:

A1. A process comprising:
(I) providing a feed mixture comprising one or more xylene isomers and optionally ethylbenzene;
(II) separating the feed mixture in a p-xylene recovery unit using simulated moving bed chromatography with toluene as a desorbent to obtain a product stream rich in p-xylene, a raffinate stream lean in p-xylene, and optionally, an intermediate stream comprising p-xylene at a concentration higher than in the raffinate stream and lower than in the product stream, the raffinate stream comprising toluene, optionally ethylbenzene, and o-xylene, m-xylene, or any combination thereof;
(III) optionally, conducting liquid-phase isomerization under liquid-phase isomerization conditions in the presence of a liquid-phase isomerization catalyst upon at least a portion of the raffinate stream to obtain an isomerized raffinate stream;
(IV) separating at least a portion of the raffinate stream and/or, if present, at least a portion of the isomerized raffinate stream in a distillation column to obtain an overhead stream comprising at least a majority of the toluene in the raffinate stream and one or more lower streams each comprising one or more xylene isomers;
(V) feeding at least a portion of the overhead stream to the p-xylene recovery unit as at least a portion of the desorbent;
(VI) conducting liquid-phase isomerization under liquid-phase isomerization conditions in the presence of a liquid-phase isomerization catalyst and obtaining one or more isomerized recycle streams after conducting the liquid-phase isomerization, the liquid-phase isomerization being conducted upon at least one of:
   (a) at least a portion of the one or more lower streams; and/or
   (b) if present, at least a portion of the intermediate stream; and/or
   (c) the liquid-phase isomerization of at least a portion of the raffinate stream is carried out in (III); and
(VII) feeding at least a portion of the one or more isomerized recycle streams to the p-xylene recovery unit.

A2. The process of A1, further comprising:
(VIII) conducting vapor-phase isomerization under vapor-phase isomerization conditions in the presence of a vapor-phase isomerization catalyst upon at least a portion of the one or more lower streams.

A3. The process of A1 or A2, wherein the liquid-phase isomerization catalyst comprises a zeolite having a MEL framework structure, a zeolite having a MFI framework structure, or any combination thereof.

A4. The process of any one of A1-A3, wherein the liquid-phase isomerization in (III) is carried out upon at least a portion of the raffinate stream to obtain the isomerized raffinate stream.

A5 The process of A4, wherein the one or more isomerized recycle streams are obtained as at least a portion of the one or more lower streams produced upon separating the isomerized raffinate stream in the distillation column in (IV).

A6. The process of A4 or A5, wherein the distillation column comprises a divided wall distillation column, and a first lower stream rich in p-xylene is obtained from a first side of the divided wall distillation column and a second lower stream lean in p-xylene is obtained from a second side of the divided wall distillation column;
wherein the one or more isomerized recycle streams are obtained as at least a portion of the first lower stream.

A7. The process of A6, further comprising:
feeding the isomerized raffinate stream to the first side of the divided wall distillation column; and
feeding at least a portion of the raffinate stream to the second side of the divided wall distillation column.

A8. The process of A4, wherein the distillation column comprises a first distillation column and a second distillation column linked in series, and the isomerized raffinate stream is fed to the first distillation column;
wherein the overhead stream comprising at least a majority of the toluene in the raffinate stream is obtained from the first distillation column, a first lower stream is obtained from the first distillation column and fed to the second distillation column, an overhead stream rich in p-xylene is obtained from the second distillation column and fed to the p-xylene recovery unit as at least a portion of the one or more isomerized recycle streams, and a second lower stream lean in p-xylene is obtained from the second distillation column.

A9. The process of A4, wherein at least a portion of the isomerized raffinate stream is fed to the p-xylene recovery unit as at least a portion of the one or more isomerized recycle streams.

A10. The process of A4 or A9, wherein at least a portion of the isomerized raffinate stream is not fed to the distillation column.

A11. The process of any one of A1-A3, wherein the liquid-phase isomerization in (VI) is carried out upon at least a portion of the one or more lower streams to produce the one or more isomerized recycle streams.

A12. The process of A11, wherein the liquid-phase isomerization in (III) is not carried out and/or the distillation column provides one lower stream.

A13. The process of any one of A1-A3, wherein the intermediate stream is obtained in (II), the liquid-phase isomerization is carried out upon at least a portion of the intermediate stream in (VI), and at least a portion of the one or more isomerized recycle streams in (VII) is obtained from the intermediate stream following liquid-phase isomerization thereon.

A14. The process of A13, wherein the intermediate stream comprises up to about 50 vol % toluene.

A15. The process of any one of A1-A14, wherein the feed mixture is obtained from a catalytic reforming process, a benzene or toluene alkylation process, a xylene isomerization process, a toluene disproportionation process, a transalkylation process, cracking, a petroleum source, a bio-production process, or any combination thereof.

A16. The process of any one of A1-A15, wherein the feed mixture and/or the isomerized recycle stream comprises 20 wt % ethylbenzene or less.

A17. The process of any one of A1-A16, wherein the raffinate stream comprises up to about 50 vol % toluene.

A18. The process of any one of A1-A17, wherein liquid-phase isomerization is conducted upon one of the intermediate stream, the raffinate stream, or the one or more lower streams.

A19. The process of any one of A1-A18, wherein the one or more isomerized recycle streams are returned to the p-xylene recovery unit at the same location or a different location than that where the feed mixture is introduced to the p-xylene recovery unit.

A20. The process of any one of A1-A19, wherein the one or more isomerized recycle streams comprise up to about 50 vol % toluene.

B1. A process comprising:
(i) providing a feed mixture comprising one or more xylene isomers and optionally ethylbenzene;
(ii) separating the feed mixture in a p-xylene recovery unit using simulated moving bed chromatography with toluene as a desorbent to obtain a product stream rich in p-xylene and a raffinate stream lean in p-xylene, the raffinate stream comprising toluene, optionally ethylbenzene, and o-xylene, m-xylene, or any combination thereof;
(iii) conducting liquid-phase isomerization under liquid-phase isomerization conditions in the presence of a liquid-phase isomerization catalyst upon at least a portion of the raffinate stream to obtain an isomerized raffinate stream:
(iv) feeding the isomerized raffinate stream to a first side of a divided wall distillation column;
(v) feeding a portion of the raffinate stream to a second side of the divided wall distillation column;
(vi) obtaining a first lower stream rich in p-xylene from the first side of the divided wall distillation column, a second lower stream lean in p-xylene from the second side of the divided wall distillation column, and an overhead stream rich in toluene from the divided wall distillation column;
(vii) feeding at least a portion of the overhead stream to the p-xylene recovery unit as at least a portion of the desorbent; and
(viii) feeding one or more isomerized recycle streams to the p-xylene recovery unit, the one or more isomerized recycle streams being obtained as at least a portion of the first lower stream.

B2. The process of B1, wherein the overhead stream comprises at least a majority of the toluene in the raffinate stream.

B3. The process of B1 or B2, wherein the liquid-phase isomerization catalyst comprises a zeolite having a MEL framework structure, a zeolite having a MFI framework structure, or any combination thereof.

B4. The process of any one of B1-B3, further comprising:
(ix) conducting vapor-phase isomerization under vapor-phase isomerization conditions in the presence of a vapor-phase isomerization catalyst upon at least a portion of the second lower stream.

C1. A process comprising:
(A) providing a feed mixture comprising one or more xylene isomers and optionally ethylbenzene;
(B) separating the feed mixture in a p-xylene recovery unit using simulated moving bed chromatography with toluene as a desorbent to obtain a product stream rich in p-xylene, a raffinate stream lean in p-xylene, and optionally an intermediate stream comprising p-xylene at a concentration higher than in the raffinate stream and lower than in the product stream, the raffinate stream comprising toluene, optionally ethylbenzene, and o-xylene, m-xylene, or any combination thereof;
(C) separating at least a portion of the raffinate stream in a distillation column to obtain an overhead stream comprising at least a majority of the toluene in the raffinate stream and one or more lower streams each comprising one or more xylene isomers;
(D) feeding at least a portion of the overhead stream to the p-xylene recovery unit as at least a portion of the desorbent;
(E) conducting liquid-phase isomerization under liquid-phase isomerization conditions in the presence of a liquid-phase isomerization catalyst to produce one or more isomerized recycle streams, the liquid-phase isomerization being conducted upon at least one of:
   (a) at least a portion of the one or more lower streams; and/or
   (b) if present, at least a portion of the intermediate stream; and/or
   (c) at least a portion of the raffinate stream; and
   (F) feeding at least a portion of the one or more isomerized recycle streams to the p-xylene recovery unit.

C2. The process of C1, wherein the liquid-phase isomerization is conducted upon at least a portion of the one or more lower streams, and at least a portion of the one or more isomerized recycle streams are obtained from the one or more lower streams after conducting the liquid-phase isomerization thereon.

C3. The process of C1, wherein the liquid-isomerization is conducted upon at least a portion of the raffinate stream to produce an isomerized raffinate stream, and at least a portion of the one or more isomerized recycle streams is obtained from the isomerized raffinate stream.

C4. The process of C1, wherein the intermediate stream is obtained, the liquid-phase isomerization is conducted upon at least a portion of the intermediate stream, and at least a portion of the one or more isomerized recycle streams is obtained from the intermediate stream after conducting the liquid-phase isomerization thereon.

C5. The process of any one of C1-C4, wherein the liquid-phase isomerization catalyst comprises a zeolite having a MEL framework structure, a zeolite having a MFI framework structure, or any combination thereof.

C6. The process of any one of C1-C5, further comprising:
(G) conducting vapor-phase isomerization under vapor-phase isomerization conditions in the presence of a vapor-phase isomerization catalyst upon at least a portion of the one or more lower streams.

To facilitate a better understanding of the embodiments of the present disclosure, the following examples of preferred or representative embodiments are given. In no way should the following examples be read to limit, or to define, the scope of the invention.

### Examples

Catalyst 1. A catalyst substantially inert to toluene conversion under liquid-phase isomerization conditions was prepared as described in U.S. Patent Application Publication 2022/0134318. The catalyst had a ZSM-11 zeolite framework with a Si:A12 ratio of 25:1. This catalyst was used to obtain the data presented in Tables 1A and 1B below.

Catalyst 2. A catalyst effective for liquid-phase isomerization but not completely inert toward toluene conversion was prepared as described in U.S. Pat. No. 4,526,879. The catalyst had a ZSM-5 zeolite framework with a Si/Ab molar ratio of approximately 26 and a crystallite size of approximately 100 nanometers. This catalyst was used to obtain the data presented in Table 2 below. In brief, the zeolite was synthesized from a mixture of n-propylamine sol, silica, aluminum sulfate sol, and aqueous NaOH aqueous solution. The uncalcined zeolite (80 parts) was mixed with HSA alumina (20 parts) and water in a muller. The resulting mixture was extruded and then dried at 121° C. overnight. The dried extrudate was calcined in nitrogen at 538° C. for 3 hours to decompose and remove the n-propylamine templating agent. Thereafter, the calcined extrudate was humidified with saturated air and exchanged with 1 N ammonium nitrate to lower the sodium level to <500 wppm. The extrudate was then washed with DI water to remove residual nitrate ions prior to drying. The extrudate was then dried at 121° C. overnight and calcined in air at 538° C. for 3 hours to obtain alumina-bound hydrogen-form ZSM-5. The catalyst extrudate had a total surface area of 450 m²/g, a hexane sorption value of 90 mg/g, and an alpha value of 900.

Liquid-phase Isomerization. A commercial raffinate stream lean in p-xylene (obtained from simulated moving bed chromatography separation of p-xylene from mixed xylenes) was mixed in various ratios with toluene and treated under liquid-phase isomerization conditions specified in Tables 1A and 1B below using Catalyst 1. Table 1A shows results with 100% raffinate, and Table 1B shows results with 1:1 wt/wt toluene/raffinate and 3:1 wt/wt toluene raffinate. Data shown in FIG. 2 below was obtained with Catalyst 2.

As shown, the liquid-phase isomerization catalyst inert toward toluene (Catalyst 1, Tables 1A and 1B) afforded less xylene loss and higher p-xylene yields than did the catalyst less inert toward toluene (Catalyst 2. Table 2).

Many alterations, modifications, and variations will be apparent to one having ordinary skill in the art in light of the foregoing description without departing from the spirit or scope of the present disclosure and that when numerical limits and numerical upper limits are listed herein, ranges from any lower limit to any upper limit are contemplated.

TABLE 1A

| Feed | 100% Raffinate | | | | | |
|---|---|---|---|---|---|---|
| Pressure (psig) | 264 | 264 | 264 | 264 | 264 | 264 |
| H₂ (ppm) | 9 | 9 | 9 | 9 | 9 | 9 |
| WHSV (hour⁻¹) | 2.5 | 5 | 10 | 15 | 2.5 | 2.5 |
| Temperature (° C.) | 240 | 240 | 240 | 240 | 220 | 200 |
| Ethylbenzene Conv. (%) | 1.5 | 1.1 | 0.9 | 0.3 | 0.3 | 0 |
| p-Xylene selectivity (wt %) | 23.69 | 23.66 | 23.21 | 22.32 | 22.78 | 16.7 |
| Benzene Yield (ppm) | 800 | 600 | 400 | 320 | 240 | 100 |
| [Raffinate Basis] | [800] | [600] | [400] | [320] | [240] | [100] |
| C9+ aromatic HC yield (ppm) | 1800 | 1270 | 820 | 585 | 427 | 138 |
| [Raffinate Basis] | [1800] | [1270] | [820] | [585] | [427] | [138] |
| Toluene yield (wt %) | 0 | 0 | 0 | 0 | 0 | 0 |
| Xylene loss (wt %) | 0.12 | 0.06 | 0.03 | 0.01 | 0 | 0 |
| [Raffinate Basis] | [0.12] | [0.06] | [0.03] | [0.01] | [0] | [0] |
| Raffinate Equiv. WSHV (hour⁻¹) | 2.5 | 5 | 10 | 15 | 2.5 | 2.5 |

TABLE 1B

| Feed | 1:1 wt/wt Toluene/Raffinate | | | 1:1 wt/wt Toluene/Raffinate | | | |
|---|---|---|---|---|---|---|---|
| Pressure (psig) | 264 | 264 | 264 | 264 | 264 | 264 | 264 |
| H₂ (ppm) | 9 | 9 | 9 | 9 | 9 | 9 | 9 |
| WHSV (hour⁻¹) | 2.5 | 5 | 10 | 2.5 | 5 | 10 | 15 |
| Temperature (° C.) | 240 | 240 | 240 | 240 | 240 | 240 | 240 |
| Ethylbenzene Conv. (%) | 1.3 | 0.8 | 0.6 | 0.8 | 0.1 | 0 | 0 |
| p-Xylene selectivity (wt %) | 23.74 | 23.76 | 23.71 | 23.78 | 23.76 | 23.71 | 23.58 |
| Benzene Yield (ppm) | 510 | 370 | 250 | 280 | 190 | 140 | 110 |
| [Raffinate Basis] | [255] | [185] | [125] | [70] | [48] | [35] | [28] |
| C9+ aromatic HC yield (ppm) | 861 | 601 | 364 | 441 | 305 | 128 | 79 |
| [Raffinate Basis] | [431] | [301] | [182] | [110] | [76] | [32] | [20] |
| Toluene yield (wt %) | 0 | 0 | 0 | 0 | 0 | 0 | 0 |
| Xylene loss (wt %) | 0.23 | 0.23 | 0.23 | 0 | 0 | 0 | 0 |
| [Raffinate Basis] | [0.115] | [0.115] | [0.115] | [0] | [0] | [0] | [0] |
| Raffinate Equiv. WSHV (hour⁻¹) | 1.25 | 2.5 | 5 | 0.625 | 1.25 | 2.5 | 3.75 |

TABLE 2

| Feed | 100% Raffinate | | | |
|---|---|---|---|---|
| Pressure (psig) | 264 | 264 | 264 | 264 |
| H₂ (ppm) | 9 | 9 | 9 | 9 |
| WHSV (hour⁻¹) | 2.5 | 5 | 7.5 | 10 |
| Temperature (° C.) | 240 | 240 | 240 | 240 |
| Ethylbenzene Conv. (%) | 1.9 | 1.3 | 1.0 | 0.8 |
| p-Xylene selectivity (wt %) | 23.49 | 22.49 | 21.04 | 19.34 |
| Benzene Yield (ppm) | 1100 | 750 | 610 | 500 |
| [Raffinate Basis] | [1100] | [750] | [610] | [500] |
| C9+ aromatic HC yield | 2420 | 1560 | 1190 | 940 |
| (ppm) | [2420] | [1560] | [1190] | [940] |
| [Raffinate Basis] | | | | |
| Toluene yield (wt %) | 0.1 | 0.066 | 0.05 | 0.036 |
| Xylene loss (wt %) | 0.23 | 0.15 | 0.11 | 0.08 |
| [Raffinate Basis] | [0.23] | [0.15] | [0.11] | [0.08] |
| Raffinate Equiv. WSHV | 2.5 | 5 | 7.5 | 10 |
| (hour⁻¹) | | | | |

All documents described herein are incorporated by reference herein for purposes of all jurisdictions where such practice is allowed, including any priority documents and/or testing procedures to the extent they are not inconsistent with this text. As is apparent from the foregoing general description and the specific embodiments, while forms of the disclosure have been illustrated and described, various modifications can be made without departing from the spirit and scope of the disclosure. Accordingly, it is not intended that the disclosure be limited thereby. For example, the compositions described herein may be free of any component, or composition not expressly recited or disclosed herein. Any method may lack any step not recited or disclosed herein. Likewise, the term “comprising” is considered synonymous with the term “including.” Whenever a method, composition, element or group of elements is preceded with the transitional phrase “comprising,” it is understood that we also contemplate the same composition or group of elements with transitional phrases “consisting essentially of,” “consisting of,” “selected from the group of consisting of,” or “is” preceding the recitation of the composition, element, or elements and vice versa.

One or more illustrative incarnations incorporating one or more invention elements are presented herein. Not all features of a physical implementation are described or shown in this application for the sake of clarity. It is understood that in the development of a physical embodiment incorporating one or more elements of the present invention, numerous implementation-specific decisions must be made to achieve the developer's goals, such as compliance with system-related, business-related, government-related and other constraints, which vary by implementation and from time to time. While a developer's efforts might be time-consuming, such efforts would be, nevertheless, a routine undertaking for those of ordinary skill in the art and having benefit of this disclosure.

Unless otherwise indicated, all numbers expressing quantities of ingredients, properties such as molecular weight, reaction conditions, and so forth used in the present specification and associated claims are to be understood as being modified in all instances by the term “about.” Accordingly, unless indicated to the contrary, the numerical parameters set forth in the following specification and attached claims are approximations that may vary depending upon the desired properties sought to be obtained by the embodiments of the present invention. At the very least, and not as an attempt to limit the application of the doctrine of equivalents to the scope of the claim, each numerical parameter should at least be construed in light of the number of reported significant digits and by applying ordinary rounding techniques.

Whenever a numerical range with a lower limit and an upper limit is disclosed, any number and any included range falling within the range is specifically disclosed, including the lower limit and upper limit. In particular, every range of values (of the form. “from about a to about b,” or, equivalently, “from approximately a to b,” or, equivalently, “from approximately a-b”) disclosed herein is to be understood to set forth every number and range encompassed within the broader range of values. Also, the terms in the claims have their plain, ordinary meaning unless otherwise explicitly and clearly defined by the patentee. Moreover, the indefinite articles “a” or “an,” as used in the claims, are defined herein to mean one or more than one of the element that it introduces.

Therefore, the present disclosure is well adapted to attain the ends and advantages mentioned as well as those that are inherent therein. The particular embodiments disclosed above are illustrative only, as the present disclosure may be modified and practiced in different but equivalent manners apparent to one having ordinary skill in the art and having the benefit of the teachings herein. Furthermore, no limitations are intended to the details of construction or design herein shown, other than as described in the claims below. It is therefore evident that the particular illustrative embodiments disclosed above may be altered, combined, or modified and all such variations are considered within the scope and spirit of the present disclosure. The embodiments illustratively disclosed herein suitably may be practiced in the absence of any element that is not specifically disclosed herein and/or any optional element disclosed herein.

What is claimed is:

1. A process comprising:
(I) providing a feed mixture comprising one or more xylene isomers and optionally ethylbenzene;
(II) separating the feed mixture in a p-xylene recovery unit using simulated moving bed chromatography with toluene as a desorbent to obtain a product stream rich in p-xylene, a raffinate stream lean in p-xylene, and optionally, an intermediate stream comprising p-xylene at a concentration higher than in the raffinate stream and lower than in the product stream, the raffinate stream comprising toluene, optionally ethylbenzene, and o-xylene, m-xylene, or any combination thereof,
(III) optionally, conducting liquid-phase isomerization under liquid-phase isomerization conditions in the presence of a liquid-phase isomerization catalyst upon at least a portion of the raffinate stream to obtain an isomerized raffinate stream;
(IV) separating at least a portion of the raffinate stream and/or, if present, at least a portion of the isomerized raffinate stream in a distillation column to obtain an overhead stream comprising at least a majority of the toluene in the raffinate stream and one or more lower streams each comprising one or more xylene isomers;
(V) feeding at least a portion of the overhead stream to the p-xylene recovery unit as at least a portion of the desorbent;
(VI) conducting liquid-phase isomerization under liquid-phase isomerization conditions in the presence of a liquid-phase isomerization catalyst and obtaining one or more isomerized recycle streams after conducting the liquid-phase isomerization, the liquid-phase isomerization being conducted upon at least one of:
(a) at least a portion of the one or more lower streams; and/or
(b) if present, at least a portion of the intermediate stream; and/or
(c) the liquid-phase isomerization of at least a portion of the raffinate stream is carried out in (III); and
(VII) feeding at least a portion of the one or more isomerized recycle streams to the p-xylene recovery unit.

2. The process of claim 1, further comprising:
(VIII) conducting vapor-phase isomerization under vapor-phase isomerization conditions in the presence of a vapor-phase isomerization catalyst upon at least a portion of the one or more lower streams.

3. The process of claim 1, wherein the liquid-phase isomerization catalyst comprises a zeolite having a MEL framework structure, a zeolite having a MFI framework structure, or any combination thereof.

4. The process of claim 1, wherein the liquid-phase isomerization in (III) is carried out upon at least a portion of the raffinate stream to obtain the isomerized raffinate stream.

5. The process of claim 4, wherein the one or more isomerized recycle streams are obtained as at least a portion of the one or more lower streams produced upon separating the isomerized raffinate stream in the distillation column in (IV).

6. The process of claim 4, wherein the distillation column comprises a divided wall distillation column, and a first lower stream rich in p-xylene is obtained from a first side of the divided wall distillation column and a second lower stream lean in p-xylene is obtained from a second side of the divided wall distillation column;
wherein the one or more isomerized recycle streams are obtained as at least a portion of the first lower stream.

7. The process of claim 6, further comprising:
feeding the isomerized raffinate stream to the first side of the divided wall distillation column; and
feeding at least a portion of the raffinate stream to the second side of the divided wall distillation column.

8. The process of claim 4, wherein at least a portion of the isomerized raffinate stream is fed to the p-xylene recovery unit as at least a portion of the one or more isomerized recycle streams.

9. The process of claim 4, wherein at least a portion of the isomerized raffinate stream is not fed to the distillation column.

10. The process of claim 1, wherein the liquid-phase isomerization in (VI) is carried out upon at least a portion of the one or more lower streams to produce the one or more isomerized recycle streams.

11. The process of claim 10, wherein the liquid-phase isomerization in (III) is not carried out and/or the distillation column provides one lower stream.

12. The process of claim 1, wherein the intermediate stream is obtained in (II), the liquid-phase isomerization is carried out upon at least a portion of the intermediate stream in (VI), and at least a portion of the one or more isomerized recycle streams in (VII) is obtained from the intermediate stream following liquid-phase isomerization thereon.

13. The process of claim 12, wherein the intermediate stream comprises up to about 50 vol % toluene.

14. The process of claim 1, wherein the feed mixture and/or the isomerized recycle stream comprises 20 wt % ethylbenzene or less.

15. The process of claim 1, wherein the raffinate stream comprises up to about 50 vol % toluene.

16. The process of claim 1, wherein liquid-phase isomerization is conducted upon one of the intermediate stream, the raffinate stream, or the one or more lower streams.

17. A process comprising:
(i) providing a feed mixture comprising one or more xylene isomers and optionally ethylbenzene;
(ii) separating the feed mixture in a p-xylene recovery unit using simulated moving bed chromatography with toluene as a desorbent to obtain a product stream rich in p-xylene and a raffinate stream lean in p-xylene, the raffinate stream comprising toluene, optionally ethylbenzene, and o-xylene, m-xylene, or any combination thereof,
(iii) conducting liquid-phase isomerization under liquid-phase isomerization conditions in the presence of a liquid-phase isomerization catalyst upon at least a portion of the raffinate stream to obtain an isomerized raffinate stream;
(iv) feeding the isomerized raffinate stream to a first side of a divided wall distillation column;
(v) feeding a portion of the raffinate stream to a second side of the divided wall distillation column;
(vi) obtaining a first lower stream rich in p-xylene from the first side of the divided wall distillation column, a second lower stream lean in p-xylene from the second side of the divided wall distillation column, and an overhead stream rich in toluene from the divided wall distillation column;
(vii) feeding at least a portion of the overhead stream to the p-xylene recovery unit as at least a portion of the desorbent; and
(viii) feeding one or more isomerized recycle streams to the p-xylene recovery unit, the one or more isomerized recycle streams being obtained as at least a portion of the first lower stream.

18. The process of claim 17, wherein the overhead stream comprises at least a majority of the toluene in the raffinate stream.

19. The process of claim 17, wherein the liquid-phase isomerization catalyst comprises a zeolite having a MEL framework structure, a zeolite having a MFI framework structure, or any combination thereof.

20. The process of claim 17, further comprising:
(ix) conducting vapor-phase isomerization under vapor-phase isomerization conditions in the presence of a vapor-phase isomerization catalyst upon at least a portion of the second lower stream.

21. A process comprising:
(A) providing a feed mixture comprising one or more xylene isomers and optionally ethylbenzene;
(B) separating the feed mixture in a p-xylene recovery unit using simulated moving bed chromatography with toluene as a desorbent to obtain a product stream rich in p-xylene, a raffinate stream lean in p-xylene, and optionally an intermediate stream comprising p-xylene at a concentration higher than in the raffinate stream and lower than in the product stream, the raffinate stream comprising toluene, optionally ethylbenzene, and o-xylene, m-xylene, or any combination thereof,
(C) separating at least a portion of the raffinate stream in a distillation column to obtain an overhead stream comprising at least a majority of the toluene in the raffinate stream and one or more lower streams each comprising one or more xylene isomers;
(D) feeding at least a portion of the overhead stream to the p-xylene recovery unit as at least a portion of the desorbent;
(E) conducting liquid-phase isomerization under liquid-phase isomerization conditions in the presence of a liquid-phase isomerization catalyst to produce one or more isomerized recycle streams, the liquid-phase isomerization being conducted upon at least one of:
(a) at least a portion of the one or more lower streams; and/or
(b) if present, at least a portion of the intermediate stream; and/or
(c) at least a portion of the raffinate stream; and
(F) feeding at least a portion of the one or more isomerized recycle streams to the p-xylene recovery unit.

22. The process of claim 21, wherein the liquid-phase isomerization is conducted upon at least a portion of the one or more lower streams, and at least a portion of the one or more isomerized recycle streams are obtained from the one or more lower streams after conducting the liquid-phase isomerization thereon.

23. The process of claim 21, wherein the liquid-isomerization is conducted upon at least a portion of the raffinate stream to produce an isomerized raffinate stream, and at least a portion of the one or more isomerized recycle streams is obtained from the isomerized raffinate stream.

24. The process of claim 21, wherein the intermediate stream is obtained, the liquid-phase isomerization is conducted upon at least a portion of the intermediate stream, and at least a portion of the one or more isomerized recycle streams is obtained from the intermediate stream after conducting the liquid-phase isomerization thereon.

25. The process of claim 21, wherein the liquid-phase isomerization catalyst comprises a zeolite having a MEL framework structure, a zeolite having a MFI framework structure, or any combination thereof.

26. The process of claim 21, further comprising:
(G) conducting vapor-phase isomerization under vapor-phase isomerization conditions in the presence of a vapor-phase isomerization catalyst upon at least a portion of the one or more lower streams.
